(19) United States
(12) Patent Application Publication
(10) Pub. No.: US 2025/0297003 A1
(43) Pub. Date: Sep. 25, 2025

(54) INHIBITING MAST CELL ACTIVATION BY BINDING SIALIC ACID-BINDING IMMUNOGLOBULIN-LIKE LECTIN-9 (SIGLEC-9)

(71) Applicant: Seattle Children’s Hospital d/b/a Seattle Children’s Research Institute, Seattle, WA (US)

(72) Inventors: Adrian Piliponsky, Seattle, WA (US); Nyssa Becker Samanas, Seattle, WA (US); Irina Miralda Molina, Seattle, WA (US)

(73) Assignee: Seattle Children’s Hospital d/b/a Seattle Children’s Research Institute, Seattle, WA (US)

(21) Appl. No.: 18/863,540

(22) PCT Filed: May 5, 2023
(86) PCT No.: PCT/US2023/066710

§ 371 Date: Nov. 6, 2024

Related U.S. Application Data
(60) Provisional application No. 63/339,317, filed on May 6, 2022.
(60) Provisional application No. 63/488,752, filed on Mar. 6, 2023.

(51) Int. Cl. C07K 16/28

(52) U.S. Cl. CPC C07K 16/2803; C07K 16/283; C07K 2317/73; C07K 2317/76; C07K 2317/77

(57) ABSTRACT

Inhibiting mast cell activation by binding sialic acid-binding immunoglobulin-like lectin-9 (Siglec-9) is described. Inhibiting mast cell activation by binding Siglec-9 can be used to treat mast-cell associated inflammatory disorders, such as allergic diseases, rheumatoid arthritis, and mastocytosis.

### CROSS REFERENCE TO RELATED APPLICATIONS

This application is a U.S. National Phase Application based on International Patent Application No. PCT/US2023/066710, filed on May 5, 2023, which claims priority to U.S. Provisional Patent Application No. 63/488,752, filed Mar. 6, 2023, and claims priority to U.S. Provisional Patent Application No. 63/339,317, filed May 6, 2022, the entire contents of which are incorporated herein by reference.

### REFERENCE TO SEQUENCE LISTING

The Sequence Listing associated with this application is provided in xml format in lieu of a paper copy, and is hereby incorporated by reference into the specification. The name of the text file containing the Sequence Listing is 3BM5302.XML. The text is 24,576 bytes, was created on Oct. 17, 2024, and is being submitted electronically via Patent Center.

### FIELD OF THE DISCLOSURE

The current disclosure provides methods and compositions for inhibiting mast cell activation by binding sialic acid-binding immunoglobulin-like lectin-9 (Siglec-9) on mast cells. Inhibiting mast cell activation by binding Siglec-9 can be used to treat mast-cell associated inflammatory disorders, such as allergic diseases, rheumatoid arthritis, and mastocytosis.

### BACKGROUND OF THE DISCLOSURE

Mast cells are hematopoietic progenitor-derived, granule-containing immune cells that are widely distributed in tissues that interact with the external environment, such as the skin and mucosal tissues. They are characterized by large granules that store inflammatory mediators such as histamine, heparin, cytokines, and proteases. Mast cells have been proposed to contribute to defense against pathogens, wound healing, and tumor surveillance.

While mast cells have a number of beneficial physiological effects, their activation is also associated with a number of inflammatory disorders. For example, numerous preclinical and clinical studies recognize mast cells as key effector cells in urticaria, mastocytosis and allergic disease.

Sialic acid-binding immunoglobulin-like lectins (Siglec(s)) are I-type lectins that are expressed by a number of cells including cells of the hematopoietic system. The Siglecs include a number of families of molecules, each characterized by the presence of a N-terminal V-set Ig-like domain, which mediates sialic acid binding, followed by varying numbers of C2-set Ig-like domains.

### SUMMARY OF THE DISCLOSURE

The current disclosure provides methods and compositions for inhibiting mast cell activation by binding sialic acid-binding immunoglobulin-like lectin-9 (Siglec-9) on mast cells. Inhibiting mast cell activation by binding Siglec-9 can be used to treat mast-cell associated inflammatory disorders, such as allergic diseases, rheumatoid arthritis, and mastocytosis.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1A. Depiction of mast cell expressing high affinity receptor for immunoglobin E (IgE) and degranulation of the mast cell following antigen binding to IgE.

FIG. 1B illustrates pS9L, a Siglec-9 agonist including a lactosyl polypeptide conjugated to a sialic acid, and including a cellular membrane anchor.

FIG. 2. Siglec-9 is not expressed on murine mast cells.

FIG. 3. Siglec-9 expression on human cells. A portion of this data is also presented in FIGS. 21A and 22A.

FIG. 4. Expression of Siglecs on human mast cell lines. This data is also presented in FIGS. 21B and 23A.

FIG. 5. Glycophorin A inhibits mast cell degranulation.

FIG. 6. High molecular weight hyaluronic acid (HMW-HA) inhibition of degranulation.

FIG. 7. Clustered Regularly Interspaced Short Palindromic Repeats (CRISPR) editing of LAD2 cells.

FIG. 8. Siglec-9 deficient mast cells show higher activation at baseline.

FIG. 9. Siglec-9 deficient mast cells are hyperreactive to various stimuli.

FIG. 10. Inhibition LAD2 degranulation by Siglec-9 ligands.

FIG. 11. Siglec-9 agonistic monoclonal antibody (mAb) specifically inhibits degranulation of wildtype (WT) LAD2.

FIG. 12. Siglec-9 expression in human lung mast cells.

FIG. 13. Siglec-9 expression by peripheral blood cultured mast cells (PBCMC).

FIG. 14. Siglec-9 agonist inhibition of PBCMC degranulation.

FIG. 15. Siglec-9 expression during mast cells differentiation.

FIG. 16. Gradual change in media from Media I for hematopoietic cell expansion (Iscove's Modified Dulbecco's Medium (MDM) with bovine serum albumin, recombinant human insulin, human transferrin, 2-mercaptoethanol, supplements, interleukin (IL)-6, stem cell factor (SCF), and ciprofloxacin) to Media II for mast cell (progenitor) expansion (Iscove's MDM with GlutaMAX and bovine serum albumin, recombinant human insulin, human transferrin, 2-mercaptoethanol, IL-6, SCF, and ciprofloxacin).

FIG. 17. Culturing PBCMC with Media I restores Siglec-9 expression.

FIG. 18. Culture with IgE (4 days) increases Siglec-9 expression (cells cultured in media II).

FIG. 19. FCεRI^{-/-} PBCMC do not upregulate Siglec-9 after IgE culture.

FIG. 20. Culture with Media I or IgE induces maximal expression of Siglec-9.

FIGS. 21A, 21B. Siglec-7, Siglec-8, and Siglec-9 surface expression in human mast cell lines. (21A, 21B) Representative flow cytometry analysis of Siglec-9 surface expression in HMC-1.2, LAD2 and LUVA cells (21A) and percentage of HMC-1.2, LAD2 and LUVA cells expressing Siglec-7, Siglec-8, and Siglec-9 (21B). Flow cytometry data in (21A) is representative of 3-5 experiments. Data in (21B) are shown as mean+SEM with circles showing values from individual experiments. **** P<0.0001.

FIGS. 22A-22F. Siglec-9 expression in human primary mast cells. (22A, 22B) Representative flow cytometry analysis of Siglec-9 surface expression in human neutrophils, human peripheral blood mononuclear cell-derived mast cells (PBCMCs), human skin cultured mast cells (HSCMCs), and human lung mast cells (22A) and percentage of PBCMCs, HSCMCs and human lung mast cells expressing Siglec-7, Siglec-8, and Siglec-9 (22B). (22C) Kinetics of Siglec-9 surface expression on CD34⁺-derived PBCMCs. (22D) Representative confocal microscopy images show intracellular and cell surface staining for Siglec-9 in PBCMCs (green fluorescence in right panel). Nuclei were counterstained with DAPI (blue fluorescence). Negative control was performed with secondary antibodies only (left panel). Scale bar equals 10 μm. (22E, 22F) Mean fluorescence intensity (MFI) of Siglec-9 in PBCMCs (22E) and HSCMCs (22F) stimulated with an anti-FcεRIα antibody (100 ng/ml) or maintained in medium alone for 20 min. Flow cytometry data in (22A) and confocal images in (22D) are representative of 2-6 experiments. Data in (22B), (22C), (22E) and (22F) are shown as mean+SEM with circles in (22B), triangles in (22E) and squares in (22F) showing values from individual experiments with cells generated from individual donors. * P<0.05, ** P<0.01, *** P<0.001, **** P<0.0001.

FIGS. 23A-23C. SIGLEC7, SIGLEC8, and SIGLEC9 mRNA expression in human mast cells. Messenger RNA expression levels for the indicated transcripts are expressed as ΔCT values normalized against GAPDH as the reference transcript. Data are shown as mean+SEM of the average for duplicate specimens. Circles in (23A) show values from individual experiments with human mast cell lines. Circles in (23B) and (23C) show values from individual experiments with cells generated from individual donors.

FIG. 24. Siglec-9 localization in LAD2 cells. Representative confocal microscopy images show intracellular and cell surface staining for Siglec-9 in LAD2 cells (green fluorescence in right panel). Nuclei were counterstained with DAPI (blue fluorescence). Negative control was performed with secondary antibodies only (left panel). Scale bar equals 10 μm. Confocal images are representative of 2 experiments.

FIGS. 25A-25F. Siglec-9 is internalized following antibody ligation. (25A and 25D) Representative flow cytometry analysis of surface (25A) and total (25D) Siglec-9 expression in HSCMCs treated with 5 μg/ml of either isotype control (Iso) or anti-Siglec-9 antibody (S9) for the indicated time points. Mean fluorescence intensity (25B and 25E) and percentage of signal lost (25C and 25F) at indicated points for surface (25B and 25C) and total (25E and 25F) Siglec-9 expression in HSCMCs treated with 5 μg/ml of either isotype control or anti-Siglec-9 antibody (Anti-Sig9) for the indicated time points. The percentage of signal lost was calculated as MFI for Siglec-9 at indicated point minus MFI for Siglec-9 at time 0. Flow cytometry data in (25A) and (25D) are representative of 3-5 independent experiments. Data in (25B), (25C), (25E) and (25F) are shown as mean+SEM with squares showing values from individual experiments with cells generated from individual donors. * P<0.05, ** P<0.01, **** P<0.0001.

FIGS. 26A-26F. Siglec-9 internalization in LAD2 cells following antibody ligation. (26A and 26D) Representative flow cytometry analysis of surface (26A) and total (26D) Siglec-9 expression in LAD2 cells treated with 5 μg/ml of either isotype control (Iso) or anti-Siglec-9 antibody (S9) for the indicated time points. Mean fluorescence intensity (26B and 26E) and percentage of signal lost (26C and 26F) at indicated points for surface (26B and 26C) and total (26E and 26F) Siglec-9 expression in LAD2 cells treated with 5 μg/ml of either isotype control or anti-Siglec-9 antibody (Anti-Sig9) for the indicated time points. The percentage of signal lost was calculated as MFI for Siglec-9 at indicated point minus MFI for Siglec-9 at time 0. (26G) Representative confocal microscopy images show intracellular and cell surface staining for Siglec-9, and intracellular staining for Rab5 and Rab7 in LAD2 cells treated with 5 μg/ml of either isotype control (Iso) or anti-Siglec-9 antibody (S9) for the indicated time points. Nuclei were counterstained with DAPI (blue fluorescence). Siglec-9 (green fluorescence) co-localization with Rab 5 and Rab7 (red fluorescence) is indicated with white arrowheads. Scale bar equals 10 μm. Flow cytometry data in (26A) and (26D) are representative of 3 experiments. Confocal images in (26G) are representative of 2 experiments. Data in (26B), (26C), (26E) and (26F) are shown as mean+SEM from with circles showing values from individual experiments with LAD2 cells. * P<0.05, ** P <0.01, **** P<0.0001.

FIGS. 27A-27K. Siglec-9 interactions with sialic acids in cis limit mast cell activation. (27A) Representative flow cytometry analysis of Siglec-9 expression of mock- and SIGLEC9-edited LAD2 cells. (27B) Siglec surface expression in mock- and SIGLEC9-edited deficient LAD2 cells. (27C) LAMP-1 surface expression in unedited and SIGLEC9-edited deficient LAD2 cells maintained in medium alone. (27D-27F) β-hexosaminidase release by unedited and SIGLEC9-edited LAD2 cells upon activation. Unedited and SIGLEC9-edited cells were sensitized with IgE (2 μg/ml) overnight and then were challenged with either anti-human IgE (500 ng/ml) (27D), compound 48/80 (c48/80) (10 μM) (27E), or calcium ionophore (A23187) (1 μm) (27F) for 1 h. (27G-27I) Representative flow cytometry analysis of Siglec-9 ligand expression (27G), percentage (27H) of HSCMCs expressing Siglec-9 ligands. (27I) Representative fluorescent microscopy images show cell surface binding of Fc chimera Siglec-9 protein in HSCMCs (green in right panel). Nuclei were counterstained with DAPI (red fluorescence). Negative control was performed with secondary antibodies only (left panel). Scale bar equals 10 μm. (27J-27K) LAMP-1 expression in unedited and SIGLEC9-edited LAD2 cells (27J), and HSCMCs maintained in medium alone or treated with sialidases (10 mU/ml) for 1 h. Flow cytometry data in (27A and 27G), and microscopy images in (27I) are representative of 2-3 independent experiments. Data in (27B-27F, 27H and 27J-27K) are shown as mean+SEM. Circles in (27B-27F and 27J) show values from individual experiments with LAD2 cells. Squares in (27H and 27K) show values from individual experiments with cells generated from individual donors. * P<0.05, ** P<0.01, *** P<0.001, **** P<0.0001.

FIGS. 28A, 28B. Expression of Siglec-9 ligands in LAD2 cells. (28A, 28B) Representative flow cytometry analysis of Siglec-9 ligand expression (28A) and percentage (28B) of unedited and SIGLEC9-edited LAD2 cells expressing Siglec-9 ligands. Unedited LAD2 cells were maintained in medium alone or treated with sialidases (10 mU/ml) for 1 h. Flow cytometry data in (28A) is representative of 3 experiments. Data in (28B) are shown as mean+SEM with circles showing values from individual experiments with LAD2 cells. * P<0.05, ** P<0.01, *** P<0.001, **** P <0.0001.

FIGS. 29A-29F. Expression of sialyltransferases and sialic acid biosynthesis enzymes in human mast cells. Messenger RNA expression levels for GNE (29A), ST3GAL1 (29B), ST3GAL3 (29C), ST3GAL4 (29D), ST3GAL5 (29E), ST3GAL6 (29F) are shown. Messenger RNA expression levels for the indicated transcripts are expressed as ΔCt values normalized against GAPDH as the reference transcript. Data are shown as mean+SEM of the average for duplicate specimens. Triangles and squares show values from individual experiments with cells generated from individual donors.

FIGS. 30A, 30B. Siglec-9 ligands inhibit mast cell degranulation. (30A, 30B) Inhibition of β-hexosaminidase release in LAD2 cells (30A) and reduction in LAMP-1 expression in PBCMCs (30B) treated with either glycophorin A (GlycA) (25-100 μg/ml) or high molecular weight hyaluronic acid (HMW-HA) (20-100 μg/ml) for 20 min before stimulation. For LAD2 cell IgE-dependent activation, cells were sensitized with IgE (2 μg/ml) overnight and then were challenged with anti-human IgE (500 ng/ml) for 1 h. For PBCMC IgE-dependent activation, cells were treated with anti-FcεRIα antibodies (100 ng/ml) for 20 min. For IgE-independent mast cell activation, LAD2 cells and PBCMCs were stimulated with compound 48/80 (c48/80) (10 μM) for 1 h and 20 min, respectively. Data are shown as mean+SEM. Circles in (30A) show values from individual experiments with LAD2 cells. Triangles in (30B) show values from individual experiments with cells generated from individual donors. * P<0.05, ** P<0.01, *** P<0.001, **** P<0.0001 vs. cells treated with stimuli alone.

FIG. 31. Siglec-9 ligands do not inhibit SIGLEC9-edited LAD2 cell degranulation. β-hexosaminidase release in SIGLEC9-edited LAD2 cells treated with either glycophorin A (GlycA) (25 μg/ml) or high molecular weight hyaluronic acid (HMW-HA) (50 μg/ml) for 20 min before stimulation. SIGLEC9-edited LAD2 cells were sensitized with IgE (2 μg/ml) overnight and then were challenged with anti-human IgE (500 ng/ml) for 1 h. Data are shown as mean+SEM with circles showing values from individual experiments with LAD2 cells. **** P<0.0001.

FIGS. 32A-32E. Siglec-9 engagement with an anti-Siglec-9 antibody inhibits LAD2 cell but not human primary mast cell degranulation. (32A, 32B) β-hexosaminidase release in LAD2 cells sensitized with IgE (2 μg/ml) overnight and then challenged with either anti-human IgE (500 ng/ml) (32A) or compound 48/80 (c48/80) (10 μM) (32B) for 1 h. (32C-32E) LAMP-1 expression in PBCMCs (32C and 32E) and HSCMCs (32D) treated with anti-FcεRIα antibodies (100 ng/ml) for 20 min. For Siglec-9 engagement, cells were pre-incubated with either isotype control or mouse anti-Siglec-9 (5 μg/ml) prior to activation. In (32E), PBCMCs incubated with anti-Siglec-9 or isotype control were exposed to 5 μg/ml goat anti-mouse IgG (Fc specific) F(ab′)2 fragment antibody (5 μg/ml) for 2 min to cross-link Siglec-9 prior to activation. Data are shown as mean+SEM. Circles in (32A) and (32B) show values from individual experiments with LAD2 cells. Triangles in (32C) and (32E) and squares in (32D) show values from individual experiments with cells generated from individual donors. * P<0.05, **** P<0.0001.

FIG. 33. PBCMC viability in cells treated with anti-Siglec-9 antibodies or isotype control conditions at 0, 1, 24, and 48 hours.

FIGS. 34A-34F. Co-engagement of FcεRI and Siglec-9 inhibits mast cell degranulation, production of arachidonic acid metabolites, and IL-8 release. (34A-34F) LAMP-1 expression in PBCMCs (34A) and HSCMCs (34B), and reduction in LAMP-1 expression (34C), cys-LT (34D), PGD₂ (34E) and IL-8 production in PBCMCs and HSCMCs maintained in medium alone or incubated with either isotype control or mouse anti-Siglec-9 (5 μg/ml) and stimulated with anti-human FcεRIα (100 ng/ml) and a goat anti-mouse IgG (Fc specific) F(ab′)2 fragment antibody (5 μg/ml) to cross-link FcεRIα and Siglec-9. Data are shown as mean+SEM. Triangles in (34A) and (34C-34F), and squares in (34B-34F), show values from individual experiments with cells generated from individual donors. In (34A and 34B): * P<0.05, ** P<0.01, **** P<0.0001. In 34C-34F: * P<0.05, ** P<0.01, *** P<0.001 vs. cells treated with isotype control.

FIGS. 35A, 35B. (35A) Siglec-E expression in bone marrow-derived cultured mast cells (BMCMCs), fetal skin-derived cultured mast cells (FSCMCs), and peritoneal mast cells (PMCs). (35B) Siglec-E gene expression levels in blood neutrophils and mast cells from the peritoneum, esophagus, trachea, tongue, and skin.

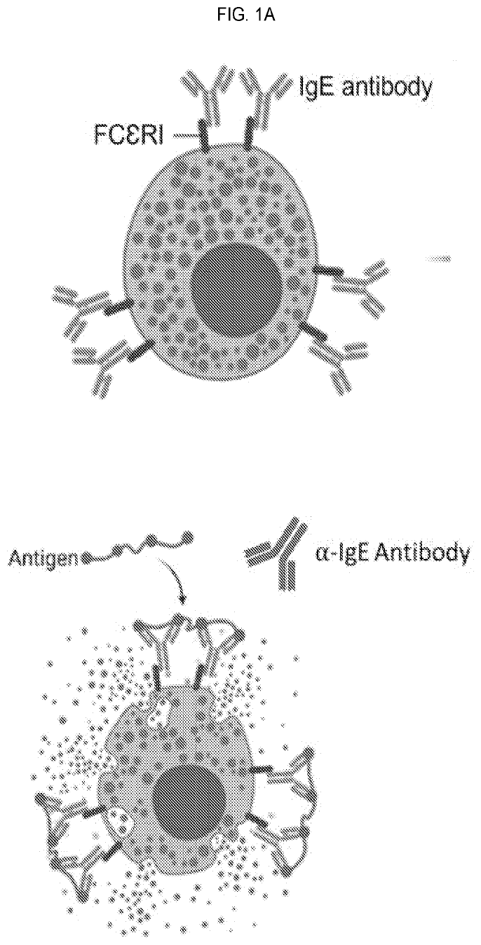

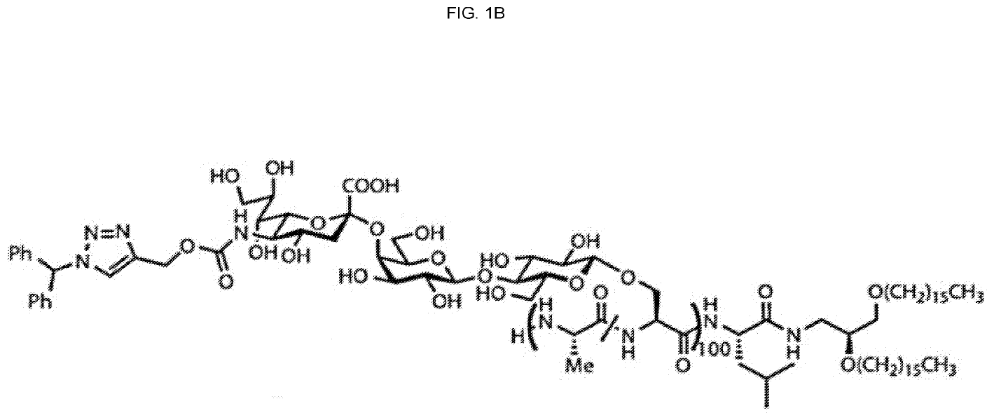

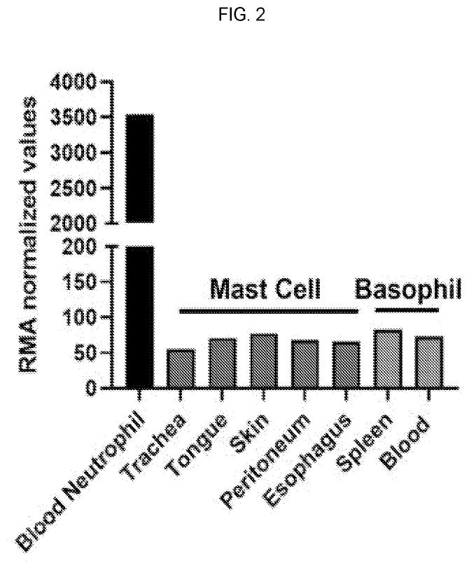

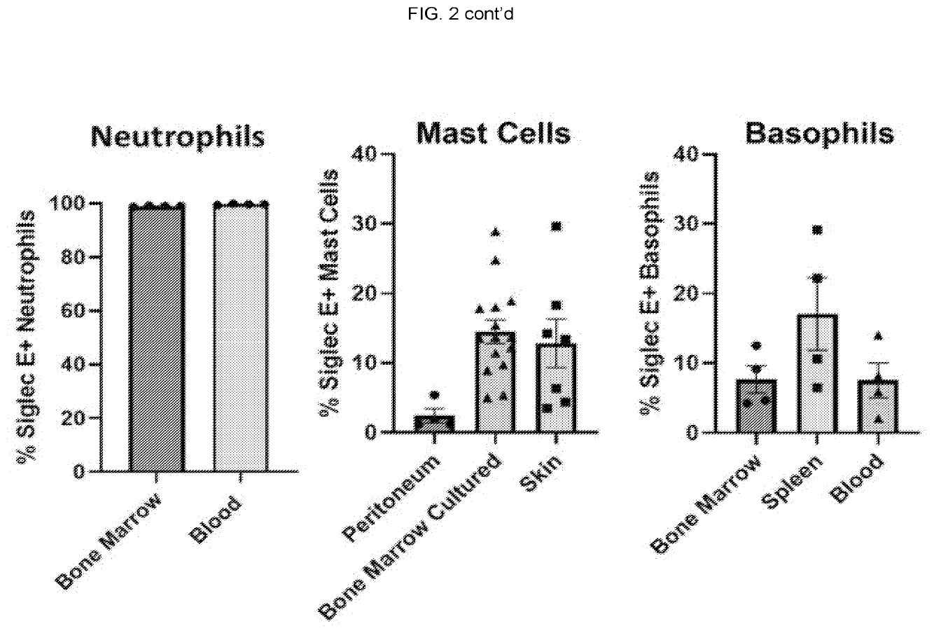

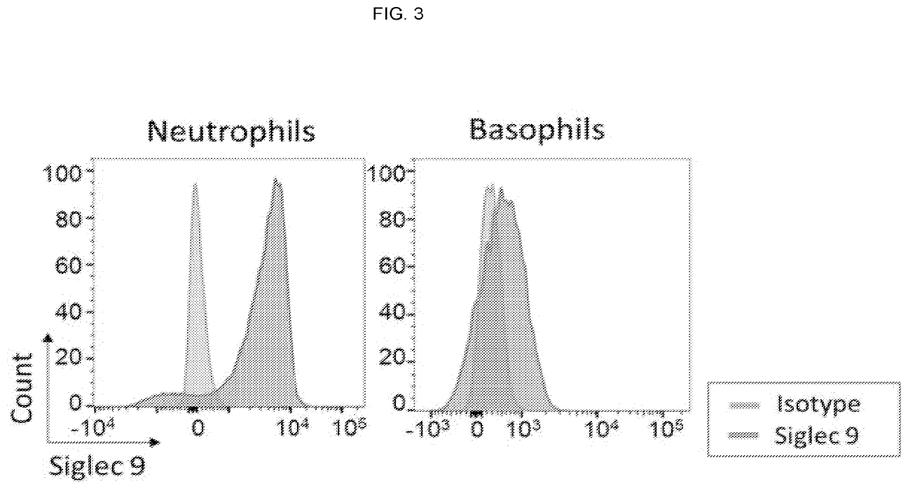

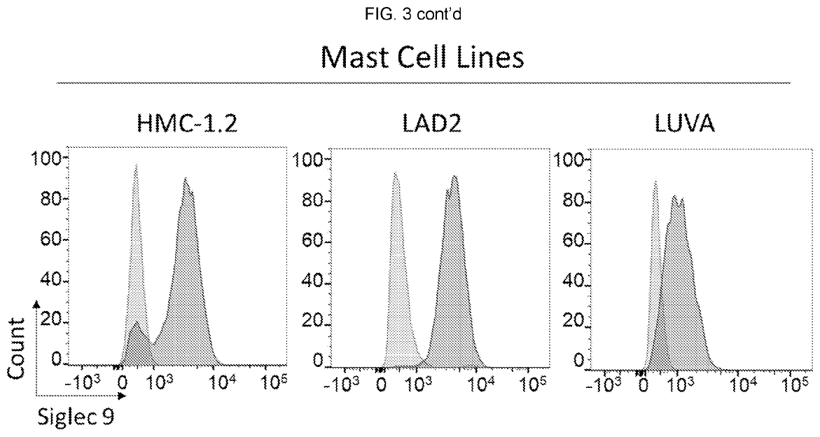

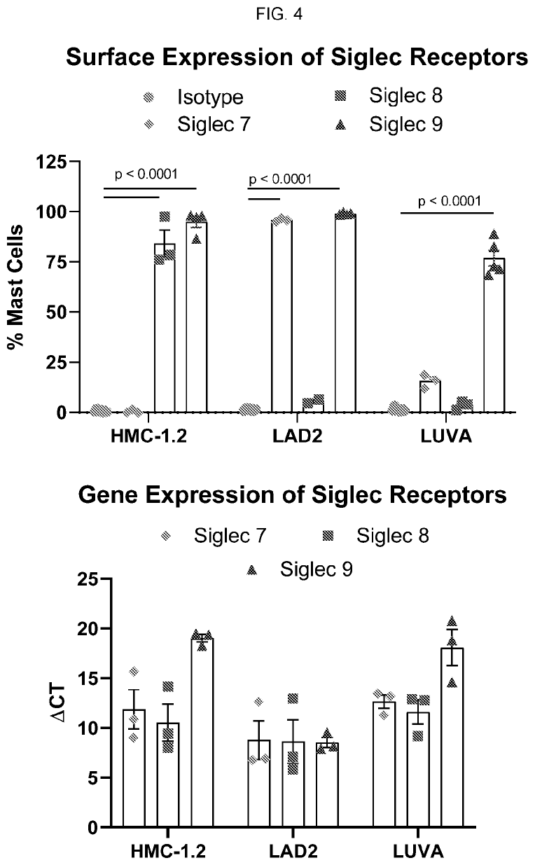

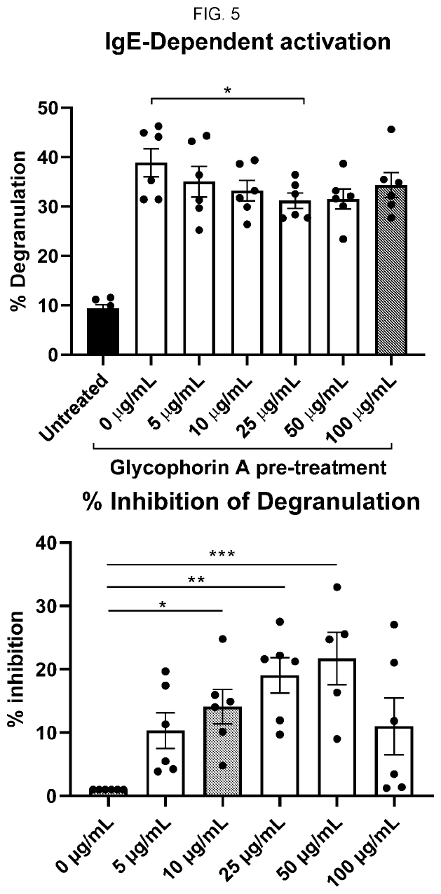

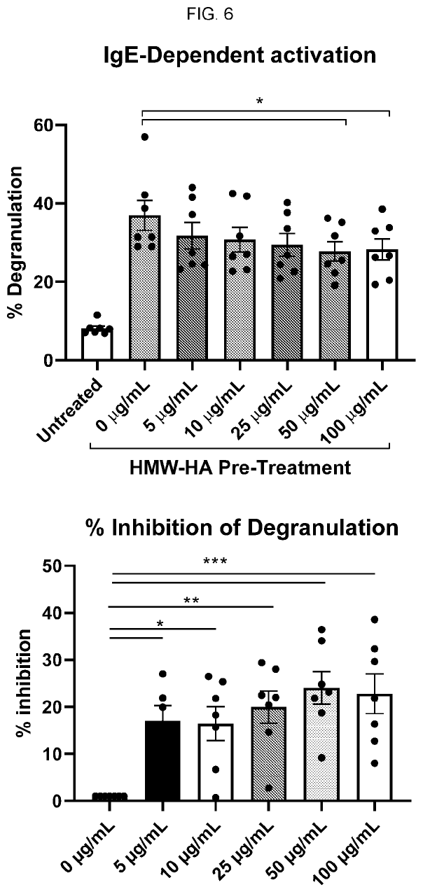

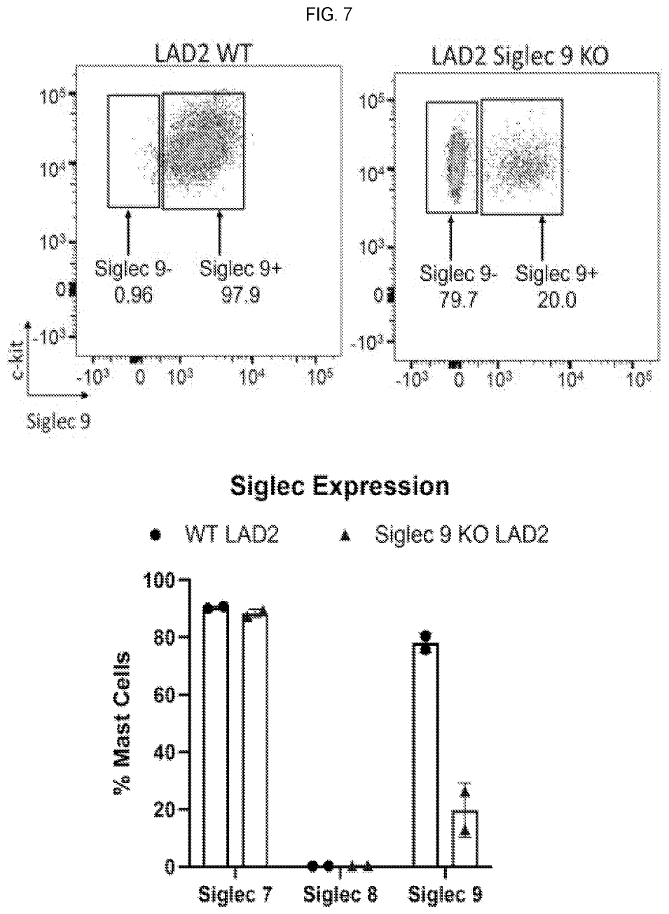

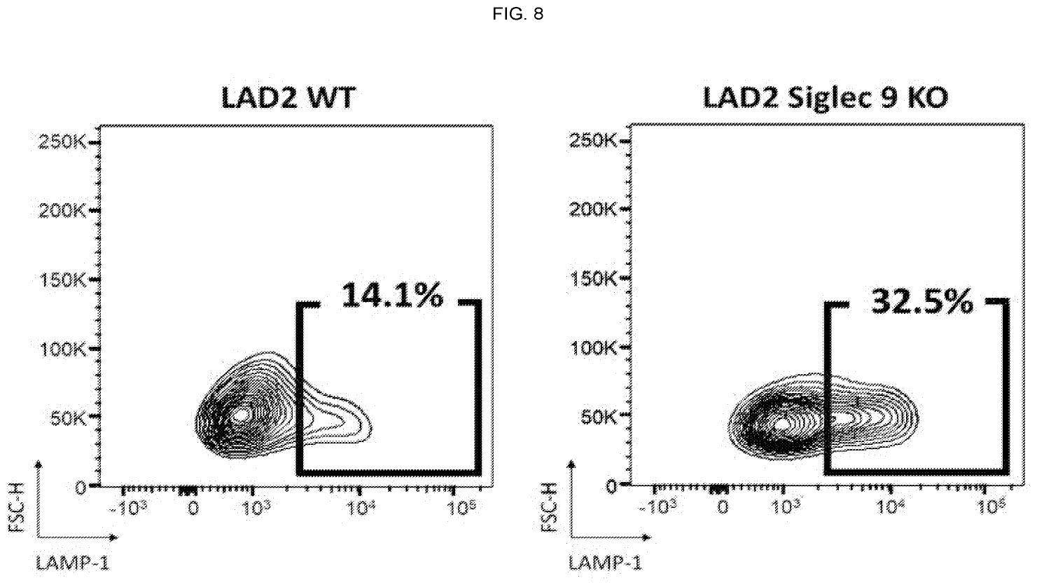

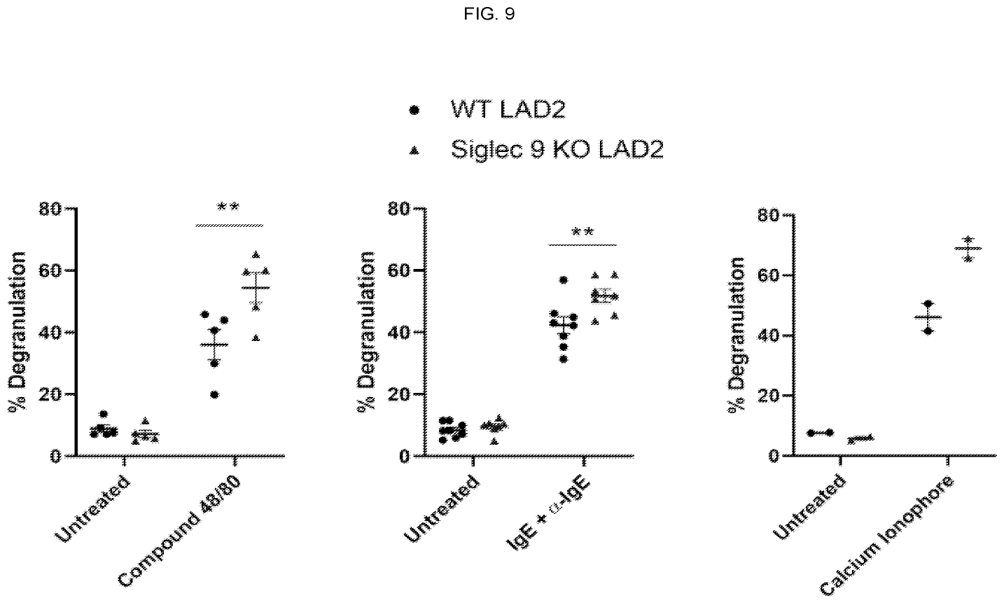

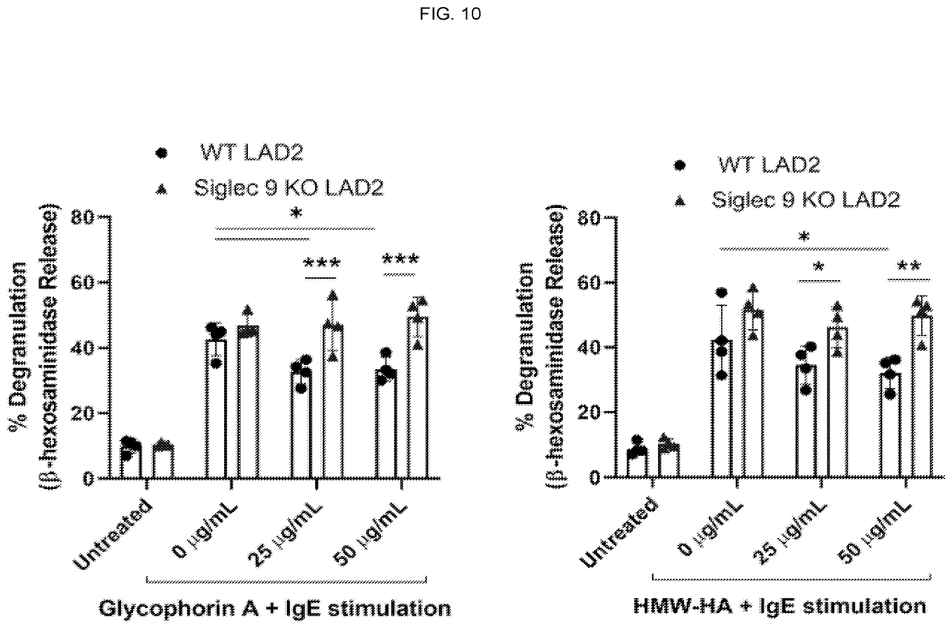

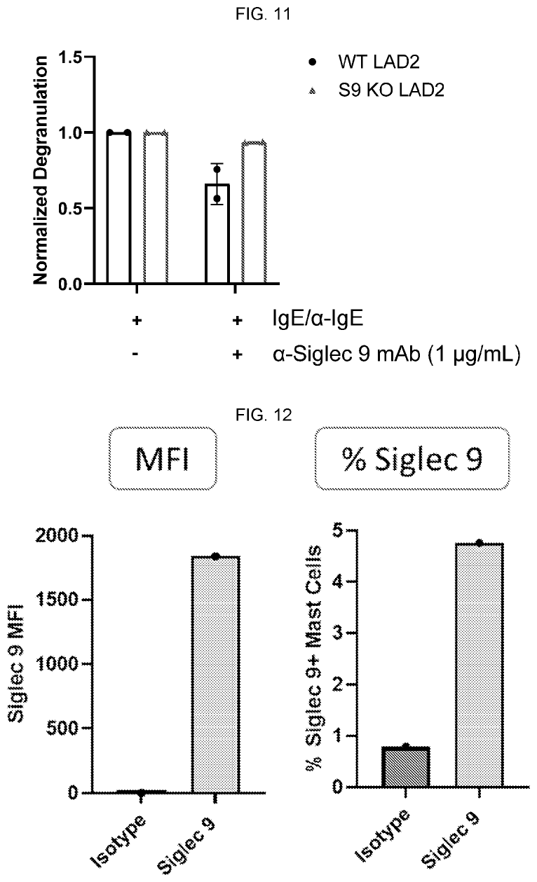

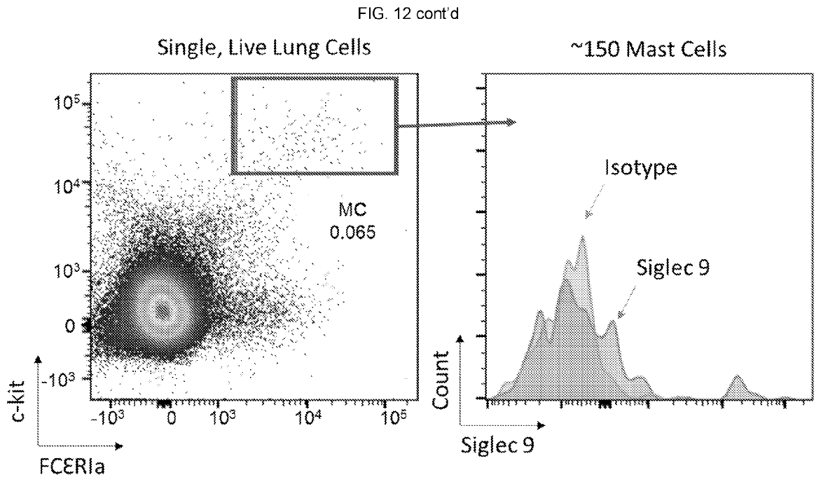

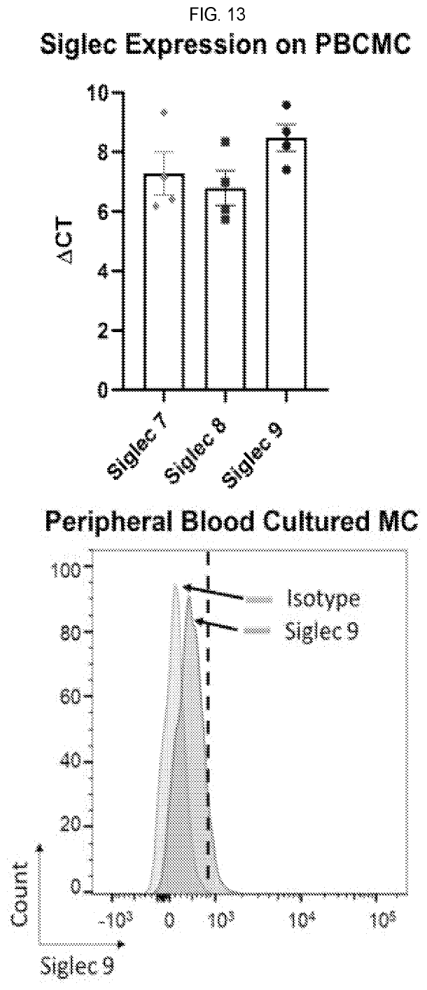

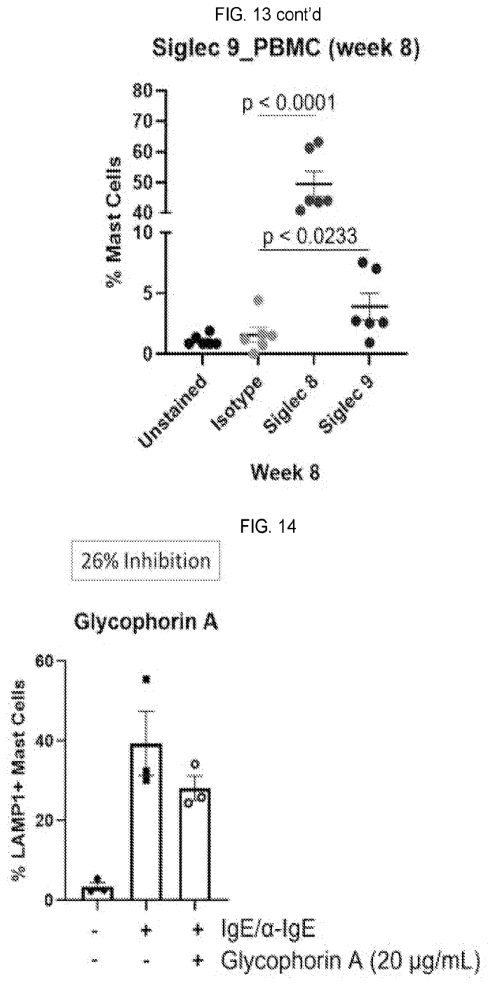

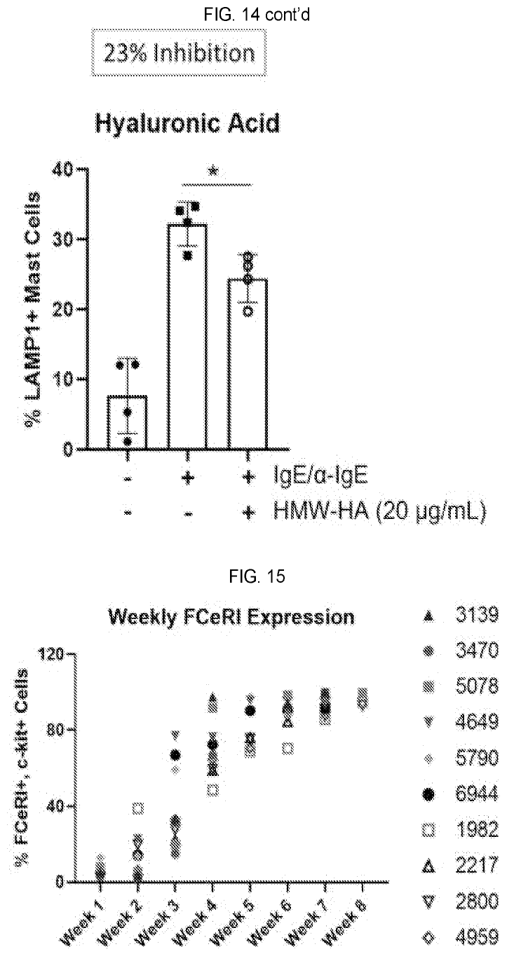

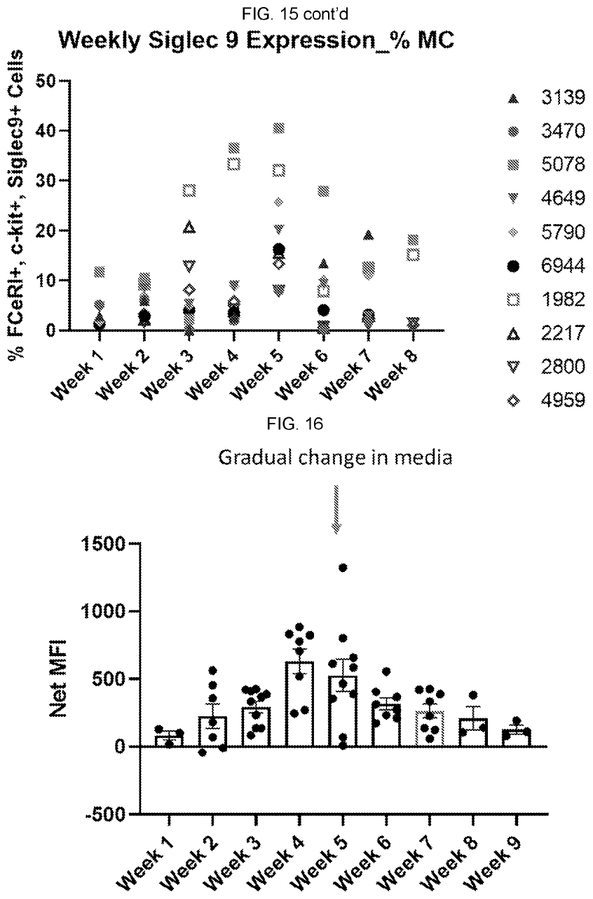

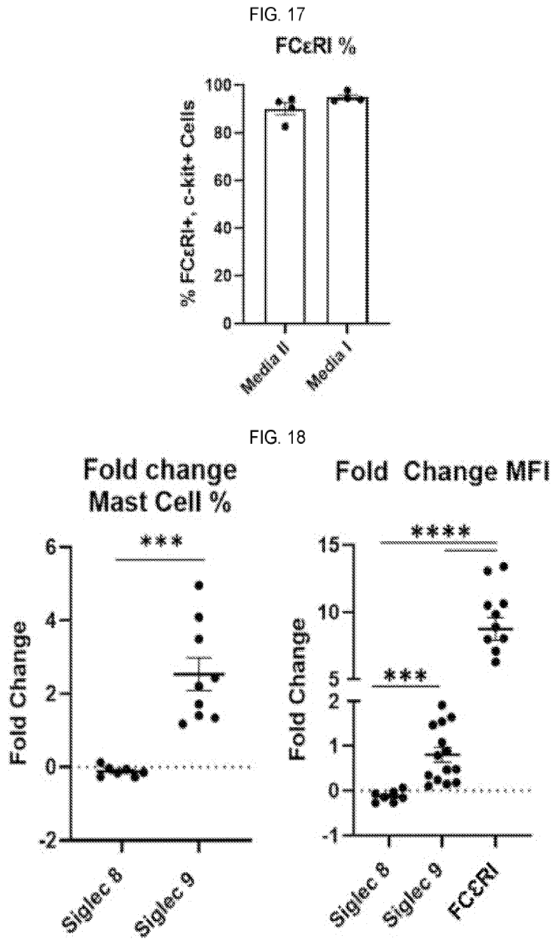

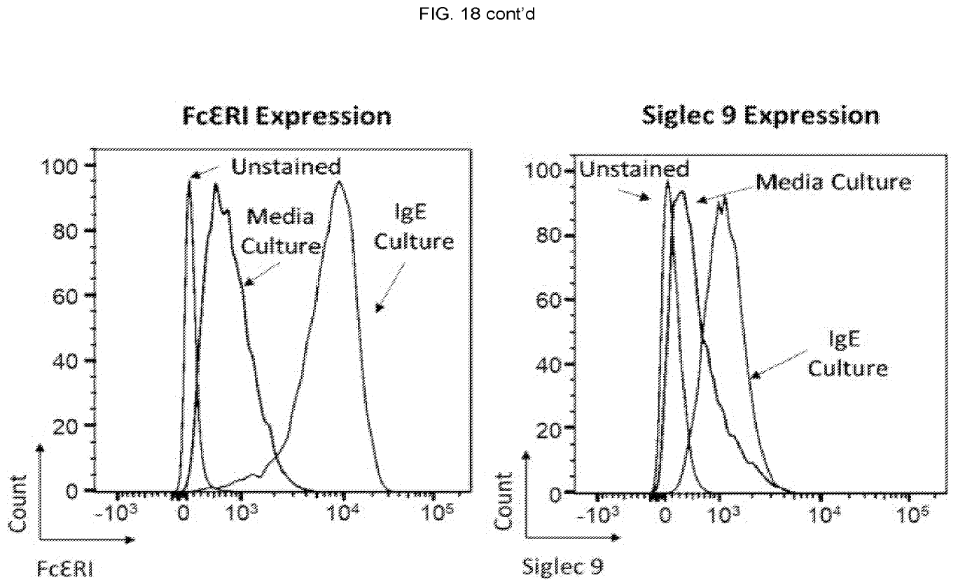

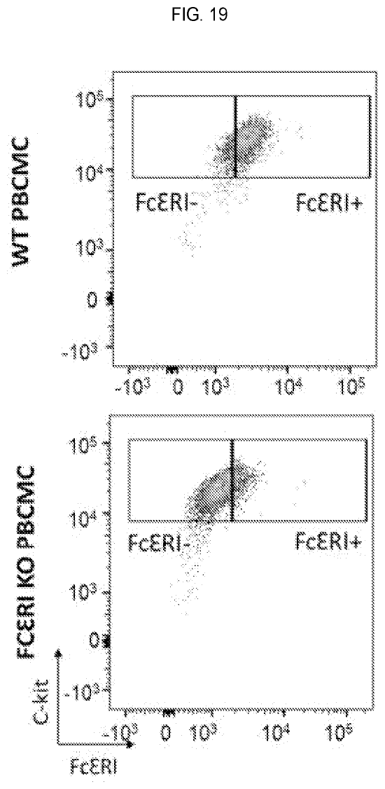

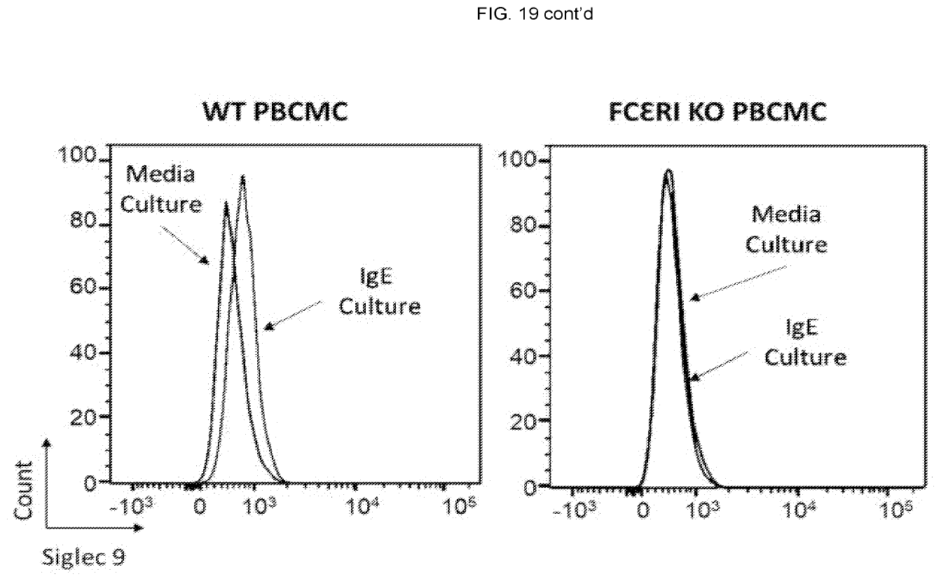

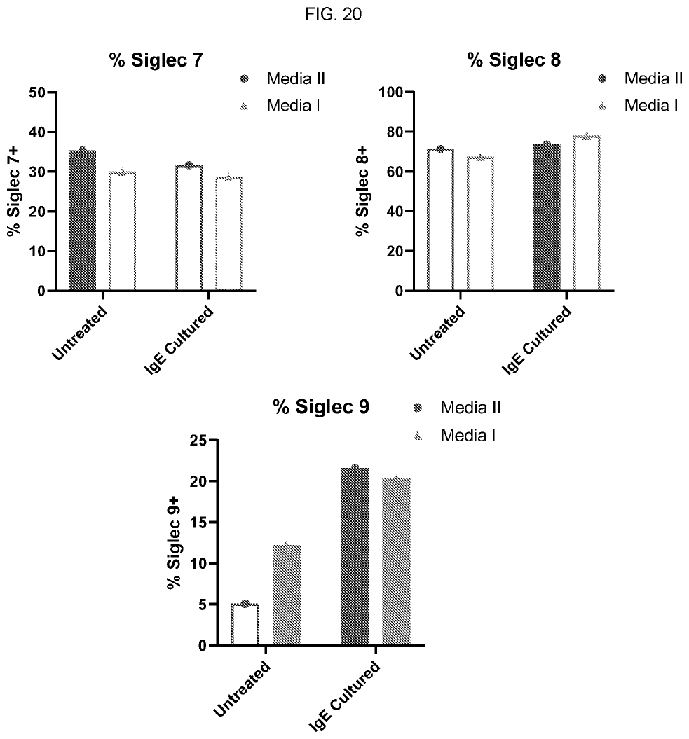

### DETAILED DESCRIPTION

Sialic acid binding immunoglobulin-like lectins (Siglecs) are cell surface transmembrane inhibitory receptors that recognize sialic acids. Sialic acids function as self-associated molecular patterns (SAMP) and suppress immune cell activation by binding to Siglecs. Pathogens and tumor cells enhance their expression of sialic acids to dampen immune responses.

The current disclosure shows that human mast cells express Siglec-9, an inhibitory immunomodulatory receptor that is mainly expressed by innate immune cells such as hematopoietic neutrophils and monocytes. The current disclosure shows that Siglec-9 is expressed in the human mast cell lines LAD2, LUVA, and HMC-1, and in peripheral blood-derived cultured human mast cells (PBCMCs). The expression of Siglec-9 in PBCMCs peaks at week 5 of culture and correlates positively with the expression of the high affinity receptor for IgE (FcεRI). Siglec-9 expression is upregulated in PBCMCs at 5 days after addition of IgE to mast cell cultures suggesting that Siglec-9 may counterbalance stimulatory signals in allergic patients that exhibit increased IgE levels. Whether Siglec-9 is functional in mast cells was assessed by using Siglec-9 agonists (e.g., agonistic Siglec-9 antibodies, glycophorin A, and high molecular weight hyaluronic acid (HMW-HA)).

Both agonistic Siglec-9 antibodies and pre-treatment of human mast cells with the Siglec-9 agonists, followed by FcεRI-dependent stimulation, had an inhibitory effect on mast cell degranulation. Moreover, Siglec-9 ligation also inhibited mast cell activation by IgE-independent mechanisms indicating that Siglec-9 can downregulate mast cell function in allergic and non-allergic conditions. SIGLEC9 gene disruption by CRISPR/Cas9 editing resulted in a significant reduction in Siglec-9 expression in LAD2 cells that also became impervious to inhibition by Siglec-9 agonists. This result confirms that Siglec-9 agonists have an inhibitory effect on mast cell activation by binding to Siglec-9. Together, the data presented herein show that human mast cells express Siglec-9 and that engaging this inhibitory receptor can reduce mast cell degranulation.

Siglec-9 deletion by a CRISPR-Cas9 approach significantly increased the expression of activation markers on mast cells at baseline and mast cell ability to undergo a more robust activation when compared to unedited cells. Mast cells exhibited a marked reduction in mast cell degranulation when Siglec-9 was engaged with native ligands prior to IgE-dependent and IgE-independent activation. Furthermore, co-aggregating Siglec-9 with FcεRI resulted in decreased degranulation and reduced production of arachidonic acid metabolites and chemokines.

Aspects of the current disclosure are now described in more supporting detail as follows: (I) Siglec-9 and Siglec-9 Ligands; (II) Compositions for Administration; (III) Methods of Use; (IV) Exemplary Embodiments; (V) Experimental Example; and (VI) Closing Paragraphs. These headings are provided for organizational purposes only and do not limit the scope or interpretation of the disclosure.

(I) Siglec-9 and Siglec-9 Ligands. Sialic-acid-binding immunoglobulin-like lectins (Siglecs) are type 1 membrane proteins having an amino-terminal V-set immunoglobulin domain and C2-set immunoglobulin domains. The V-set immunoglobulin domain mediates sialic-acid recognition. Siglecs are usually found on the surface of immune cells such mast cells, macrophages, B cells, neutrophils, monocytes, myeloid progenitors, and eosinophils. Siglecs can be divided into two groups based on sequence similarity and evolutionary conservation. The CD33-related Siglecs share high sequence similarity in their extracellular regions and often include conserved tyrosine-based signaling motifs in intracellular domains. By contrast, there are orthologues, in all mammals examined, of sialoadhesin, CD22, myelin-associated glycoprotein (MAG) and Siglec-15 and they exhibit lower sequence similarity (Crocker et al., 2007, Nature Review Immunology 7:255-266).

Siglec-9 is a member of the Siglec family highly related to Siglec-7. When expressed at the cell surface, Siglec-9 exhibits sialic acid-dependent binding to human red blood cells and synthetic sialoglycoconjugates (such as sialyl oligosaccharides conjugated to a glycoprotein), and is a putative adhesion molecule that mediates sialic-acid dependent binding to cells.

Siglec-9 expression on several types of immune cells can inhibit anti-tumor immune responses as a result to binding to sialoglycans presented by cancer cells. CD8+ T cells express Siglec-9, causing CD8+ T cell functionality and immune response to be susceptible to inhibition by cancer cells. Transcriptomic analyses of immune cells from severe COVID-19 patients show that neutrophils upregulate Siglec-9.

Given that Siglec-9 is both an anti-inflammatory and pro-apoptotic checkpoint molecule, engagement of Siglec-9 could simultaneously inhibit proinflammatory cell death and induce quiet apoptotic cell death in COVID-19-related inflammation.

Siglec-9 protein sequences are publicly available, for example, see Accession Nos: Q9Y336.2, NP_055256.1, NP_001185487.1, XP_047294571.1, XP_011525034.1, AAF71455.1, AAG23261.1, and AAF87223.1.

Accession No. XP_011525034.1 provides isoform X1:

(SEQ ID NO: 1)
MLLLLLPLLWGRERAEGQTSKLLTMQSSVTVQEGLCVHVPCSFSYPSHGWIYPGPVVHGYWF
REGANTDQDAPVATNNPARAVWEETRDRFHLLGDPHTKNCTLSIRDARRSDAGRYFFRMEKG
SIKWNYKHHRLSVNVTALTHRPNILIPGTLESGCPQNLTCSVPWACEQGTPPMISWIGTSVSPL
DPSTTRSSVLTLIPQPQDHGTSLTCQVTFPGASVTTNKTVHLNVSYPPQNLTMTVFQGDGTVS
TVLGNGSSLSLPEGQSLRLVCAVDAVDSNPPARLSLSWRGLTLCPSQPSNPGVLELPWVHLR
DAAEFTCRAQNPLGSQQVYLNVSLQSKATSGVTQGVVGGAGATALVFLSFCVIFVVVRSCRKK
SARPAAGVGDTGIEDANAVRGSASQKTSSSSSSAFEQLFPHPSIPFRAEASKYYLSGTDRNYF
RLHGPHSLIAAPQICCCSKKEAIYPV.

while Accession No. XP_047294571.1 provides isoform X2:

(SEQ ID NO: 2)
MLLLLLPLLWGRERAEGQTSKLLTMQSSVTVQEGLCVHVPCSFSYPSHGWIYPGPVVHGYWF
REGANTDQDAPVATNNPARAVWEETRDRFHLLGDPHTKNCTLSIRDARRSDAGRYFFRMEKG
SIKWNYKHHRLSVNVTALTHRPNILIPGTLESGCPQNLTCSVPWACEQGTPPMISWIGTSVSPL
DPSTTRSSVLTLIPQPQDHGTSLTCQVTFPGASVTTNKTVHLNVSYPPQNLTMTVFQGDGTVS
TVLGNGSSLSLPEGQSLRLVCAVDAVDSNPPARLSLSWRGLTLCPSQPSNPGVLELPWVHLR
DAAEFTCRAQNPLGSQQVYLNVSLQSKATSGVTQGVVGGAGATALVFLSFCVIFVVVRSCRKK
SARPAAGVGDTGIEDANAVRGSASQGPLTEPWAEDSPPDQPPPASARSSVGEGELQYASLSF
QMVKPWDSRGQEATDTEYSEIKIHR.

Siglec-9 ligands bind to or otherwise associate with Siglec-9, and may include, for example, small organic molecules, peptides, carbohydrates and antibodies.

The Siglec-9 ligand may include the natural ligand for Siglec-9, or, a fragment, analogue or portion thereof. For example the Siglec-9 ligand may include a sialyl oligosaccharide, i.e., a carbohydrate which further includes sialic acid at a terminal end. Such oligosaccharides can include, for example, triaose, tetraose, pentose, hexose, and the like, and can be singly sialylated or disialylated.

Additional native Siglec-9 ligands include glycophorin A and high molecular weight hyaluronic acid (HMW-HA). Glycophorin A is the most abundant sialoglycoprotein on erythrocytes (sialoglycoproteins being proteins glycosylated with sialyl oligosaccharide sidechains, including the glycophorin family and podocalyxin). Glycophorin A binds to neutrophils via Siglec-9, and maintains neutrophil quiescence in the bloodstream (Lizcano et. al, Blood, 2017). HMW-HA is a highly enriched and widely distributed glycosaminoglycan (glycosaminoglycans being polysaccharide chains composed of repeating disaccharide units, such as heparan sulfates (HSGAGs), dermatan sulfate (CSGAGs), keratan sulfate, and hyaluronic acid) that is on vertebrate cells and extracellular matrices. It exists in a native high molecular weight (>1,000 kDa) and binds to neutrophils through Siglec-9 (Secundino et. al, J Mol Med, 2017).

The Siglec-9 agonist, pS9L, is described in Delaveris et al., Proc. Natl. Acad. Sci. USA 2021 Jan. 19; 118 (3): e2012408118, as a lipid-conjugated glycopolypeptide. pS9L includes a lactosyl polypeptide, conjugated to a modified sialic acid residue which demonstrates specific cis-binding to Siglec-9. pS9L can also include a cellular membrane anchor.

Mucins are highly glycosylated proteins which are components of mucus secretions from mucous membranes of various tissues. Mucin glycosylation can include glycosidically bound sialic acids, which can ligate with specificity to one or more Siglecs. By way of example, in human upper airway tissues, Siglec-9 can bind to the glycans of the mucin MUC5B (Jia et al., J Allergy Clin Immunol, 135:799-810 e7, 2015), which expresses in the respiratory tract. By way of another example, Siglec-9 can bind to the glycans of the mucins MUC1 and MUC16, each of which is expressed by cancer cells. Such Siglec-9 binding mucins are characterized by high levels of glycosylation, and particularly by O-sialoglycosylation, wherein 40% to 80% of side chains of the protein are composed of high numbers of O-linked sialylated glycans. Mucins expressed by cancer cells are characterized by short and unbranched chains.

Siglec-9 antibodies are described in U.S. Pat. No. 9,265,826 (e.g., KALLI; see also Zhang et al., J Biol. Chem. 2000; 275:22121-22126). Siglec-9 antibodies may bind various epitopes of Siglec-9, including an alpha-2,3- or an alpha-2,6-linked sialic acid, and can be expressed as a monoclonal antibody (mAb), can be expressed as a polyclonal antibody (pAb), and can be expressed as a recombinant monoclonal antibody. Exemplary Siglec-9 antibodies include clone 191240, clone K8, and KALLI.

Siglec-9 antibodies also include fragments of antibodies that retain the ability to bind or otherwise associate with Siglec-9. Advantageously therefore, the term “antibody” may include whole antibody molecules or fragments thereof which specifically bind to or otherwise associate with Siglec-9. Antibodies may readily be fragmented, for example F (ab) 2 fragments (e.g., generated by treating an antibody with pepsin) such as hS9-Fab03. F (ab) 2 fragments may be treated to reduce disulfide bridges to produce Fab fragments. Antibody fragments also include single chain variable fragments (scFv). An scFv is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

Other techniques allow antibodies to be further fragmented such that they may comprise solely the complimentary determining region(s) (CDR) of the molecule. Such antibody fragments may be known in the art as “domain antibodies”. Single domain nanobodies may also be used.

In particular embodiments, murine Sigle-9 antibodies can be humanized. A humanized antibody is an engineered antibody in which the CDRs from a non-human donor antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. In particular embodiments, a humanized antibody includes humanized variable chain regions and human constant regions.

Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a variable heavy chain framework sequence and heavy chain constant region, if present, substantially from human variable heavy chain framework and human heavy chain constant region sequences. Similarly, a humanized light chain has at least one, two and usually all three CDRs entirely or substantially from a donor antibody light chain, and a variable light chain framework sequence and light chain constant region, if present, substantially from human variable light chain framework and human light chain constant region sequences. Other than nanobodies and diabodies, a humanized antibody typically includes a humanized heavy chain and a humanized light chain. A CDR in a humanized or human antibody is substantially from or substantially identical to a corresponding CDR in a non-human antibody with at least 60%, 85%, 90%, 95% or 100% of corresponding residues are identical between the respective CDRs. In particular embodiments, a CDR in a humanized antibody or human antibody is substantially from or substantially identical to a corresponding CDR in a non-human antibody when there are no more than 3 conservative amino acid substitutions in each CDR. The variable region framework sequences of an antibody chain or the constant region of an antibody are substantially from a human variable region framework sequence or human constant region respectively when at least 70%, 80%, 85%, 90%, 95% or 100% of corresponding residues are identical to reference human sequences.

Chimeric and humanized antibodies and methods of making them are reviewed, e.g., in Almagro and Fransson, Front. Biosci. 13:1619-1633, 2008, and are further described, e.g., in Riechmann et al., Nature 332:323-329, 1988; Queen et al., Proc. Nat'l Acad. Sci. USA 86:10029-10033, 1989; U.S. Pat. Nos. 5,821,337, 7,527,791, 6,982,321, and 7,087,409; Kashmiri et al., Methods 36:25-34, 2005 (describing SDR (a-CDR) grafting); Padlan, Mol. Immunol. 28:489-498, 1991 (describing “resurfacing”); Kim, et al., PLOS One 6 (5): e19867, 2011 (describing production and characterization of chimeric monoclonal antibodies); Dall'Acqua et al., Methods 36:43-60,2005 (describing “FR shuffling”); and Osbourn et al., Methods 36:61-68, 2005 and Klimka et al., Br. J. Cancer, 83:252-260, 2000 (describing the “guided selection” approach to FR shuffling). EP-B-0239400 provides additional description of “CDR-grafting”, in which one or more CDR sequences of a first antibody is/are placed within a framework of sequences not of that antibody, for instance of another antibody.

In humanized antibodies, certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids. Human framework regions that may be used for humanization include: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al., Proc. Nati. Acad. Sci. USA, 89:4285, 1992; and Presta et al., J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al., J. Biol. Chem. 271:22611-22618, 1996).

The choice of constant region can depend, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 has weak complement-dependent cytotoxicity and human IgG4 lacks complement-dependent cytotoxicity. Human IgG1 and IgG3 also induce stronger cell mediated effector functions than human IgG2 and IgG4.

Multi-domain binding molecules include bispecific antibodies which bind at least two epitopes wherein at least one of the epitopes is located on Siglec-9. Multi-domain binding molecules include trispecific antibodies which binds at least 3 epitopes, wherein at least one of the epitopes is located on Siglec-9, and so on.

Bispecific antibodies can be prepared utilizing antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

In particular embodiments, multi-domain binding molecules disclosed herein bind Siglec-9 and FceRI. Exemplary antibodies the bind FceRI include AER-37 (CRA-1) and 15.1. In particular embodiments, these antibodies can be humanized, as described elsewhere herein.

Siglec-9 ligands can be made multivalent by incorporating Siglec-9 ligands into multi-domain binding molecules. Siglec-9 ligands can also be made multivalent by incorporating Siglec-9 ligands into or onto one or more of a polymer, dendrimer, nanoparticle, or liposome. Such multivalent Siglec-9 ligands can be synthesized by, for example, techniques such as acrylate free-radical polymerization, ring-opening metathesis polymerization, TT-allyl-nickel-catalyzed coordination polymerization, and functionalization of sialoside ligands on polymer scaffolds.

Additional Siglec-9 ligands can be identified by screening, for example, peptide phage display libraries, glycopeptide libraries or FV phage display libraries.

(II) Compositions for Administration. Siglec-9 ligands can be formulated into compositions with a pharmaceutically acceptable carrier for administration to subjects. Salts and/or pro-drugs of Siglec-9 ligands can also be used.

Exemplary generally used pharmaceutically acceptable carriers include absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredient or helps to prevent denaturation or adherence to the container wall. Typical stabilizers can include polyhydric sugar alcohols; amino acids; organic sugars or sugar alcohols; sulfur-containing reducing agents; proteins such as human serum albumin, bovine serum albumin, gelatin or immunoglobulins; hydrophilic polymers such as polyvinylpyrrolidone; monosaccharides such as xylose, mannose, fructose and glucose; disaccharides; trisaccharides, and polysaccharides.

The compositions disclosed herein can be formulated for administration by, for example, injection. For injection, compositions can be formulated as aqueous solutions, such as in buffers including Hanks' solution, Ringer's solution, or physiological saline, or in culture media, such as Iscove's Modified Dulbecco's Medium (IMDM). Injectable compositions can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of active ingredients and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and compositions are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions disclosed herein can be formulated for administration by, for example, injection, infusion, perfusion, or lavage. The compositions disclosed herein can further be formulated for intravenous, intradermal, intraarterial, intranodal, intralymphatic, intraperitoneal, intralesional, intraprostatic, intravaginal, intrarectal, intrathecal, intramuscular, intravesicular, and/or subcutaneous administration and more particularly by intravenous, intradermal, intraperitoneal, intramuscular, and/or subcutaneous injection.

(III) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts, prophylactic treatments and/or therapeutic treatments.

Therapeutically effective amounts include those that provide effective amounts, prophylactic treatments and/or therapeutic treatments.

An “effective amount” is the amount of a formulation necessary to result in a desired physiological change in the subject. For example, an effective amount can reduce mast cell degranulation in a model of mast cell activation states and/or mast cell inflammatory disorders. In degranulation, mast cells release inflammatory mediators, including preformed mediators by exocytosis of cytoplasmic granules (which include histamine, proteases, chemokines, and heparin), as well as newly synthesized mediators (which include prostaglandins, thromboxanes, and leukotrienes). Effective amounts are often administered for research purposes. Effective amounts disclosed herein can cause a statistically significant effect in an animal model or in vitro assay relevant to the assessment of a disorder associated with mast cell activation (e.g., an IgE-mediated disorder). A composition can be administered in an effective amount, wherein the effective amount dampens an immune response in relation to a relevant control condition.

A “prophylactic treatment” includes a treatment administered to a subject who does not display signs or symptoms of a mast cell disorder or displays only early signs or symptoms of a mast cell disorder such that treatment is administered for the purpose of diminishing or decreasing the risk of developing the mast cell disorder further. Thus, a prophylactic treatment functions as a preventative treatment against a mast cell disorder.

A “therapeutic treatment” includes a treatment administered to a subject who displays symptoms or signs of a mast cell disorder and is administered to the subject for the purpose of diminishing or eliminating those signs or symptoms of the mast cell disorder. The therapeutic treatment can reduce, control, or eliminate the presence or activity of the mast cell disorder and/or reduce control or eliminate side effects of the mast cell disorder.

Function as an effective amount, prophylactic treatment or therapeutic treatment are not mutually exclusive, and in particular embodiments, administered dosages may accomplish more than one treatment type.

Administration of the compositions described herein can reduce the incidence of antigen-specific mast cell degranulation by at least 10%, or at least 20%, or at least 25%, or at least 30%, or at least 35%, or at least 40%, or at least 45%, or at least 50%, or at least 55%, or at least 60%, or at least 65%, or at least 70%, or at least 80%, at least 90%, at least 92%, at least 95%, at least 97%, at least 98%, or at least 99%. In some cases, the compositions describe herein can reduce the symptoms and/or incidence of antigen-specific mast cell degranulation by 100%.

In certain examples, the compositions can reduce the incidence and severity of IgE-mediated disorders or diseases. Examples of IgE-mediated disorders or diseases that can be treated include allergic rhinitis, allergic asthma, non-allergic asthma, atopic dermatitis, allergic gastroenteropathy, anaphylaxis, urticaria, food allergies, allergic bronchopulmonary aspergillosis, parasitic diseases, interstitial cystitis, hyper-IgE syndrome, ataxia-telangiectasia, Wiskott-Aldrich syndrome, athymic lymphoplasia, IgE myeloma, graft-versus-host reaction and allergic purpura. The treatment of IgE-mediated disorders or diseases can refer to the statistically significant reduction of at least one symptom of anaphylaxis or an acute allergic reaction within a 90% confidence interval, where a symptom may be one of pruritus severity, pruritus duration, erythema, angioedema, number of urticaria areas, number of erythema areas, and/or wheezing. Any statistically significant attenuation of such one or more symptoms of an IgE-mediated disorder or disease is considered to be a treatment thereof.

IgE-mediated disorders or diseases can manifest in various allergic diseases. For example, allergic ocular diseases represent some of the most common ocular diseases. The number of people afflicted with allergic ocular diseases has risen in the last decades and is now thought to affect at least 15-20% of the population. Symptoms range in severity from irritation, redness, and swelling of the conjunctiva to cataracts and vision loss.

Allergic ocular diseases encompass a number of specific clinical entities with different mechanisms of action. IgE- and non-IgE-mediated mechanisms are thought to be involved, as are multiple cytokines, chemokines, and signaling pathways (La Rosa, M. et al. (2013) Ital. J. Pediatr. 39:18). Mast cell hyperplasia and the presence of eosinophils have been observed in some forms of allergic ocular disease, such as atopic keratoconjunctivitis (Morgan, S. J. et al. (1991) Eye 5:729-735), which can lead to the development of cataracts and vision loss.

Furthermore, in some embodiments, the subject has allergic conjunctivitis. In some embodiments, the subject has seasonal allergic conjunctivitis. In some embodiments, the subject has perennial allergic conjunctivitis. In some embodiments, the subject has atopic keratoconjunctivitis. In some embodiments, the subject has vernal keratoconjunctivitis. In some embodiments, the subject has giant papillary conjunctivitis. In some embodiments, the subject uses contact lenses. In some embodiments, the subject has increased inflammation in at least a portion of the conjunctiva, as compared to an individual without an allergic ocular disease. In some embodiments, the subject has an increased number of mast cells in at least a portion of the conjunctiva, as compared to an individual without an allergic ocular disease. In some embodiments, a conjunctival scraping obtained from the subject comprises eosinophils. In some embodiments, the compositions are administrated in dosages including an amount of at least one Siglec-9 ligand as described herein, effective to treat or prevent the clinical symptoms of an IgE-mediated disorder or disease to a statistically significant extent within a 90% confidence interval.

Compositions can be administered orally, via inhalation, injection, transdermally and/or by any other appropriate administration route.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure. (IV) Exemplary Embodiments.
1. A method of reducing mast cell activation including administering an antibody or binding fragment thereof that binds sialic acid-binding immunoglobulin-like lectin-9 (Siglec-9) on the mast cell, thereby reducing mast cell activation, wherein the antibody or binding fragment thereof includes clone 191240, clone K8, or KALLI.
2. A method of reducing mast cell activation including administering a sialic acid-binding immunoglobulin-like lectin-9 (Siglec-9) ligand that binds Siglec-9 on the mast cell, thereby reducing mast cell activation.
3. The method of embodiments 1 or 2, wherein the Siglec-9 ligand includes an antibody or binding fragment thereof, a sialoglycoprotein, a glycosaminoglycan, a sialyl oligosaccharide, or a mucin.
4. The method of embodiment 3, wherein the antibody or binding fragment thereof includes a binding domain of clone 191240, clone K8, or KALLI.
5. The method of embodiments 3 or 4, wherein the antibody or binding fragment thereof is humanized.
6. The method of any of embodiments 3-5, wherein the sialoglycoprotein includes glycophorin A.
7. The method of any of embodiments 3-6, wherein the glycosaminoglycan includes hyaluronic acid (HA).
8. The method of embodiment 7, wherein the HA is a high molecular weight hyaluronic acid (HMW) HA.
9. The method of any of embodiments 3-8, wherein the glycosaminoglycan includes heparin sulfate, dermatan sulfate, or keratan sulfate.
10. The method of any of embodiments 3-9, wherein the sialyl oligosaccharide includes triaose, tetraose, pentose, or hexose.
11. The method of any of embodiments 3-10, wherein the sialyl oligosaccharide is singly or di-sialylated.
12. The method of any of embodiments 3-11, wherein the mucin includes MUC5B, MUC1, or MUC16.
13. The method of any of embodiments 2-12, wherein the Siglec-9 ligand includes pS9L.
14. The method of any of embodiments 2-13, wherein the Siglec-9 ligand is part of a multi-domain binding molecule.
15. The method of embodiment 14, wherein the multi-domain binding molecule includes an FCεR binding domain.
16. The method of embodiment 15, wherein the FCεR binding domain includes a binding domain of AER-37 (CRA-1) or 15.1.
17. The method of embodiment 16, wherein the binding domain of AER-37 (CRA-1) or 15.1 is humanized.
18. The method of any of embodiments 2-17, further including administering an antibody or binding fragment thereof that binds FCεRI.
19. The method of embodiment 18, wherein the an antibody or binding fragment thereof that binds FCεRI includes the binding domain of of AER-37 (CRA-1) or 15.1.
20. The method of embodiment 19, wherein the binding domain of AER-37 (CRA-1) or 15.1 is humanized.
21. The method of any of embodiments 2-20, wherein the Siglec-9 ligand is attached to a polymer, dendrimer, nanoparticle, or liposome.
22. The method of any of embodiments 2-21, wherein the mast cell is a human mast cell.
23. The method of any of embodiments 2-22, wherein the reducing mast cell activation reduces mast cell degranulation, arachidonic acid production, or chemokine release.
24. The method of any of embodiments 2-23, wherein the mast cell is within a subject.
25. The method of any of embodiments 24, wherein the subject is a human subject.
26. The method of any of embodiments 23-25, wherein the reducing ameliorates a symptom of a mast-cell associated inflammatory disorder.
27. The method of embodiment 26, wherein the mast-cell associated inflammatory disorder is an allergic disease, arthritis, or mastocytosis.
28. The method of any of embodiments 23-27, wherein the reducing treats an IgE-mediated disorder.
29. The method of embodiment 28, wherein the IgE-mediated disorder includes allergic rhinitis, allergic asthma, non-allergic asthma, atopic dermatitis, allergic gastroenteropathy, anaphylaxis, urticaria, food allergy, allergic bronchopulmonary aspergillosis, parasitic disease, interstitial cystitis, hyper-IgE syndrome, ataxia-telangiectasia, Wiskott-Aldrich syndrome, athymic lymphoplasia, IgE myeloma, graft-versus-host reaction, or allergic purpura.
30. The method of any of embodiments 23-29, wherein the reducing decreases antigen-specific mast cell degranulation as compared to the amount of antigen-specific mast cell degranulation under comparable conditions absent the administering.
31. A composition including a pharmaceutically acceptable carrier and a therapeutically effective amount of a Siglec-9 ligand.
32. The composition of embodiment 31, wherein the Siglec-9 ligand includes an antibody or binding fragment thereof, a sialoglycoprotein, a glycosaminoglycan, a sialyl oligosaccharide, or a mucin.
33. The composition of embodiment 32, wherein the antibody or binding fragment thereof includes a binding domain of clone 191240, clone K8, or KALLI.
34. The composition of embodiments 32 or 33, wherein the antibody or binding fragment thereof is humanized.
35. The composition of any of embodiments 32-34, wherein the sialoglycoprotein includes glycophorin A.
36. The composition of any of embodiments 32-35, wherein the glycosaminoglycan includes hyaluronic acid (HA).
37. The composition of embodiment 36, wherein the HA is a high molecular weight hyaluronic acid (HMW) HA.
38. The composition of any of embodiments 32-37, wherein the glycosaminoglycan includes heparin sulfate, dermatan sulfate, or keratan sulfate.
39. The composition of any of embodiments 32-38, wherein the sialyl oligosaccharide includes triaose, tetraose, pentose, or hexose.
40. The composition of any of embodiments 32-39, wherein the sialyl oligosaccharide is singly or di-sialylated.
41. The composition of any of embodiments 32-40, wherein the mucin includes MUC5B, MUC1, or MUC16.
42. The composition of any of embodiments 31-41, wherein the Siglec-9 ligand includes pS9L.
43. The composition of any of embodiments 31-42, wherein the Siglec-9 ligand is part of a multi-domain binding molecule.
44. The composition of embodiment 43, wherein the multi-domain binding molecule includes an FCεR binding domain.
45. The composition of embodiment 44, wherein the FCεR binding domain includes a binding domain of AER-37 (CRA-1) or 15.1.
46. The composition of embodiment 45, wherein the binding domain of AER-37 (CRA-1) or 15.1 is humanized.
47. The composition of any of embodiments 31-46, wherein the Siglec-9 ligand is attached to a polymer, dendrimer, nanoparticle, or liposome.

### (V) Experimental Example

(A) Abstract & Summary. Mast cell activation is important for the development of allergic diseases. Ligation of Sialic acid-binding immunoglobin-like lectins (Siglecs) such as CD33, Siglec-6, -7 and -8 have been shown to inhibit mast cell activation. Recent studies showed that human mast cells express Siglec-9, an inhibitory receptor also expressed by neutrophils, monocytes, macrophages, and dendritic cells. The aim of this Example was to characterize Siglec-9 expression and function in human mast cells.

The expression of Siglec-9 and Siglec-9 ligands on human mast cell lines and human primary mast cells were assessed by qPCR, flow cytometry and confocal microscopy. A clustered regularly interspaced short palindromic repeats (CRISPR)/CRISPR associated protein 9 (Cas9) gene editing approach was used to disrupt the SIGLEC9 gene. Siglec-9 inhibitory activity on mast cell function was evaluated by using native Siglec-9 ligands; glycophorin A (GlycA) and high molecular weight hyaluronic acid (HMW-HA); a monoclonal antibody against Siglec-9; and co-engagement of Siglec-9 with the high affinity receptor for IgE (FcaRI).

The results show that human mast cells express Siglec-9 and Siglec-9 ligands. SIGLEC9 gene disruption resulted in increased expression of activation markers at baseline and increased responsiveness to IgE-dependent and IgE-independent stimulation. Pre-treatment with GlycA or HMW-HA followed by IgE-dependent or independent stimulation had an inhibitory effect on mast cell degranulation. Co-engagement of Siglec-9 with FcεRI in human mast cells resulted in reduced degranulation, arachidonic acid production, and chemokine release. Thus, this study demonstrates that Siglec-9 and its ligands play an important role in limiting human mast cell activation.

(B) Introduction. Mast cells are hematopoietic progenitor-derived, granule-containing immune cells that are widely distributed in tissues that interact with the external environment, such as the skin and mucosal tissues (Piliponsky et al., Immunol Rev, 282:188-97, 2018). Mast cells contribute to defense against pathogens, wound healing, and tumor surveillance by responding to a broad array of activating signals, secreting a wide range of inflammatory mediators, and recruiting and activating immune cells (Gri et al., Front Immunol, 3:120, 2012; Varricchi et al., Int Arch Allergy Immunol, 179:247-61, 2019; Henz et al., Exp Dermatol, 10:1-10, 2001; Dahlin et al., Allergy, 77:83-99, 2022). Despite these beneficial roles, numerous preclinical and clinical studies recognize mast cells as key effector cells in urticaria, mastocytosis and allergic disease (Metcalfe D D, Blood, 112:946-56, 2008; Galli et al., Nat Med, 18:693-704, 2012; Ishizaka et al., Monogr Allergy, 18:14-24, 1983; Nadler et al., Adv Immunol, 76:325-55, 2000; Theoharides et al., N Engl J Med, 373:163-72, 2015). These studies have shown that the dysregulated expansion and/or activation of mast cells have detrimental consequences in these disorders. Therefore, there is a need for effective new strategies that inhibit mast cell function and/or deplete active mast cells for the treatment of mast cell driven disorders.

Sialic acid-binding immunoglobin-like lectins (siglecs) are a family of single-pass cell surface receptors characterized by a N-terminal domain that binds sialylated glycans (Macauley et al., Nat Rev Immunol, 14:653-66, 2014). Most Siglecs have one or multiple immunoreceptor tyrosine-based inhibitory motifs (ITIM) on the C-terminus that trigger inhibitory signals through the recruitment of tyrosine and inositol phosphatases (Ravetch et al., Science, 290:84-9, 2000). Siglecs are predominantly found on immune cells, with each cell expressing a unique combination of Siglecs that allows them to respond to distinct sialylation patterns (Gonzalez-Gil et al., Cells, 10, 2021; O'Sullivan et al., J Leukoc Biol, 108:73-81, 2020). Prior studies have shown that human mast cells express CD22/Siglec-2, CD33/Siglec-3, Siglec-5, Siglec-6, Siglec-7, Siglec-8, and Siglec-10 (Yokoi et al., Allergy, 61:769-76, 2006). Importantly, it has been shown that Siglecs can reduce the release of mast cell mediators (Robida et al., Cells, 11, 2022; Mizrahi et al., J Allergy Clin Immunol, 134:230-3, 2014; Duan et al., J Clin Invest, 129:1387-401, 2019; Yokoi et al., J Allergy Clin Immunol, 121:499-505 e1, 2008), attenuate mast cell-dependent anaphylaxis (Duan et al., J Clin Invest, 129:1387-401, 2019), limit growth of human mast cell lines in a mouse model of mastocytosis (Landolina et al., Pharmacol Res, 158:104682, 2020), ameliorate mast cell activation and inflammation in mouse models of non-allergic airway inflammation (Schanin et al., Mucosal Immunol, 14:366-76, 2021), and reduce mast cell numbers in a mouse model of eosinophilic gastrointestinal disease (Youngblood et al., JCI Insight, 4, 2019). Thus, the capability to limit mast cell activation and expansion has made Siglecs an attractive therapeutic target to downregulate mast cell function in allergic and non-allergic diseases.

Siglec-9 is an inhibitory receptor broadly expressed by neutrophils, monocytes, macrophages, dendritic cells, and subsets of B cells, T cells, and natural killer (NK) cells (Higuchi et al., Biosci Biotechnol Biochem, 80:1141-8, 2016; von Gunten et al., Blood, 106:1423-31, 2005; Avril et al., J Immunol, 173:6841-9, 2004; Zhang et al., J Biol Chem, 275:22121-6, 2000). Siglec-9 studies have been mainly focused on its detrimental effects including dampening the innate immune response to certain pathogens (Secundino et al., J Mol Med (Berl), 94:219-33, 2016; Carlin et al., Blood, 113:3333-6, 2009; Khatua et al., J Leukoc Biol, 91:641-55, 2012; Saha et al., mBio, 12, 2021) and impairing immune surveillance in certain cancers (Jandus et al., J Clin Invest, 124:1810-20, 2014; Rodriguez et al., Nat Commun, 12:1270, 2021; Laubli et al., Proc Natl Acad Sci USA, 111:14211-6, 2014; Haas et al., Cancer Immunol Res, 7:707-18, 2019).

However, recent studies support the use of Siglec-9 engagement to inhibit excessive immune cell activation and limit the magnitude of the inflammatory response during arthritis (Matsumoto et al., Arthritis Res Ther, 18:133, 2016), colitis (Kang et al., Int Immunopharmacol, 86:106695, 2020), and severe COVID-19 (Delaveris et al., ACS Cent Sci, 7:650-7, 2021).

Siglec-9 expression and function in mast cells has been largely unexplored. Initial reports using human peripheral blood mononuclear cell derived-cultured mast cells (PBCMCs) showed very low expression of Siglec-9 mRNA by a high throughput approach (Yokoi et al., Allergy, 61:769-76, 2006). More recently, Siglec-9 was detected in human skin (Duan et al., J Clin Invest, 129:1387-401, 2019) and lung (Ronnberg et al., Front Immunol, 12:804812, 2021) mast cells. Here, it was confirmed that human mast cell lines and human primary mast cells express Siglec-9. Kinetics of Siglec-9 surface expression showed that Siglec-9 expression paralleled the expression of the high affinity receptor for IgE (FcaRI) during mast cell differentiation. Based on this evidence, further investigation was conducted on the functional relevance of Siglec-9 expression on human mast cells. Siglec-9 deletion by a clustered regularly interspaced short palindromic repeats (CRISPR)/CRISPR associated protein 9 (Cas9) approach significantly increased the expression of activation markers on mast cells at baseline, and mast cell ability to undergo a more robust activation when compared to unedited cells. Importantly, mast cells exhibited a marked reduction in mast cell degranulation when Siglec-9 was engaged with native ligands prior to IgE-dependent and IgE-independent activation. Furthermore, co-aggregating Siglec-9 with FcεRI resulted in decreased degranulation and reduced production of arachidonic acid metabolites and chemokines.

(C) Methods. Ethics statement. All animal experiments were approved by the controlling Institutional Animal Care and Use Committee (Protocol #IACUC00020) and performed in strict accordance with the recommendations in the Guide for the Care and Use of Laboratory Animals of the National Institutes of Health (8th Edition).
(1) Mice. C57BL/6J mice were purchased from Jackson Laboratories, bred, and vivarium-maintained. Mice with Siglec-E-deficiency (McMillan et al., Blood, 121:2084-94, 2013) on the C57BL/6 background were also obtained.
(2) Generation of bone marrow derived-cultured mast cells (BMCMCs). Femoral bone marrow cells from C57BL/6J mice were maintained in vitro for 6 weeks in medium containing 10 ng/ml recombinant mouse (rm) interleukin (IL)-3 (Peprotech, Rocky Hill, NJ) until the mast cells represented >95% of the total cells defined as c-Kit and FcεRIα positive cells when analyzed by flow cytometry.
(3) Generation of fetal skin derived-cultured mast cells (FSCMCs). Fetal skin cells from C57BL/6J mice were obtained as described in Yamada et al., J Invest Dermatol, 121:1425-32, 2003. These cells were maintained in vitro for 4-6 weeks in medium containing 10 ng/ml rmIL-3 and 10 ng/ml rm stem cell factor (SCF) (Peprotech) until the mast cells represented >95% of the total cells defined as c-Kit and FcεRIα positive cells when assessed by flow cytometry.
(4) Human mast cell lines. The human MCL-derived cell line HMC-1, subclone HMC-1.2 harboring KIT V560G and KIT D816V was obtained (Butterfield et al., Leuk Res, 12:345-55, 1988) and grown in Iscove's modified Dulbecco Media (IMDM) supplemented with 25 mM 4-(2-hydroxyethyl)-1-piperazineethanesulfonic acid (HEPES), 10% heat-inactivated fetal bovine serum (FBS), 2 mM L-glutamine, and 1% penicillin-streptomycin. LUVA cells (Laidlaw et al., J Allergy Clin Immunol, 127:815-22 e1-5, 2011) were obtained and maintained in StemPro-34 serum-free medium supplemented with StemPro-34 nutrient supplement (catalog number 10639011, Thermo Fisher Scientific, Waltham, MA), 2 mM L-glutamine, and 1% penicillin/streptomycin. LAD2 cells were obtained from the Laboratory of Allergic Diseases, National Institute of Allergy and Infectious Diseases, National Institutes of Health, Bethesda, MD (Kirshenbaum et al., Blood, 94:2333-42, 1999) and maintained in the same media as LUVA cells with the addition of 100 ng/ml recombinant human (rh) SCF.
(5) Human primary mast cells. Human peripheral blood was obtained from anonymous donors to Bloodworks Northwest (Seattle, WA) in accordance with established institutional guidelines and mast cells were generated as described in Folkerts et al. Nat Protoc 2020; 15:1285-310. Briefly, CD34+ hematopoietic progenitors were isolated from the peripheral blood mononuclear fraction by immunomagnetic positive selection (catalog number 17856, StemCell Technologies, Vancouver, BC, Canada). CD34+ cells were cultured in StemSpan SFEM II media (StemCell Technologies) supplemented with 10 μg/ml ciprofloxacin (Sigma-Aldrich, St. Louis, MO), 50 ng/ml rhIL-6 (PeproTech), and 3% vol/vol of conditioned medium from Chinese hamster ovary (CHO) cell transfectants secreting murine SCF for at least 8 weeks. During the first week of culture, 50 ng/ml rhIL-3 (Peprotech) was added to the culture media. A mature mast cell population containing at least 90% c-Kit and FcεRIα positive cells as assessed by flow cytometry, was observed after 5 weeks of culture. Human skin derived-cultured mast cells (HSCMCs) were generated from human skin mast cells enriched from discarded tissues that were collected at the time of mastectomies. Mast cell isolation procedures were approved by the controlling Human Subjects Review Committee. Skin mast cells were harvested and cultured as previously described (Caslin et al., Methods Mol Biol, 1799:81-92, 2018). Briefly, the subcutaneous fat was removed from skin specimens and the resultant skin was cut into small pieces and enzymatically digested in Hanks balanced salt solution (HBSS) containing 10% FBS, 10 mM HEPES, 0.035% sodium bicarbonate, 0.5% amphotericin B and 1% penicillin/streptomycin (Thermo Fisher Scientific), and supplemented with 0.7 mg/ml hyaluronidase (Sigma-Aldrich), 592.5 U/ml collagenase type II (Thermo Fisher Scientific), 0.15 mg/ml DNase type I (Sigma-Aldrich), and 1 mM CaCl₂) (Sigma-Aldrich). The specimens were digested on a shaker at 37° C. for 1 h. The digested mixture was filtered through a 70-μm cell strainer, and the remaining tissue was collected for two additional digestions. Collected cells were washed, spun down, filtered through a 40-μm filter, and washed again. The collected pellets from the three digestions were combined and layered over 75% Percoll in HBSS cushion and centrifuged at 800×g at room temperature for 20 min. Nucleated cells were collected from the buffer/Percoll interface, washed, and plated at a concentration of 5 ×10⁵ cells/ml in serum-free X-Vivo 15 media (catalog number 04-418Q iLonza, Basel, Switzerland) supplemented with 100 ng/ml rhSCF (Peprotech). Mast cells were used after 8 weeks of culture when purity was greater than 90% as assessed by positive staining for c-Kit and FcεRIα and flow cytometry.

Lung mast cells were enriched from healthy tissues provided by the National Disease Research Interchange (NDRI) program as previously described (Okayama et al., J Immunol Methods, 169:153-61, 1994; Ravindran et al., Front Immunol, 9:2193, 2018). Briefly, lung fragments of 0.5-2.0 mm³ were washed twice with Dulbecco's Modified Eagle Medium (DMEM) containing 2% FCS (DMEM/FCS) before incubation in the same buffer (1 g tissue/4 ml buffer) containing 1.5 mg/ml collagenase type IA, 0.75 mg/ml hyaluronidase, and 0.02 mg/ml DNAse. The specimens were digested on a shaker at 37° C. for 1 h. The digested mixture was filtered through a 70-μm cell strainer and washed twice with DMEM/FCS. The filtered cell suspension was resuspended in 30% Percoll (one phase) and centrifuged at 780×g for 12 min. Excess Percoll was removed by washing three times in HBSS containing 2% FCS. Siglec-9 expression was assessed by flow cytometry in the enriched lung mast cell population identified as c-Kit and FcεRIα positive cells.
(6) Human neutrophils. Nucleated cells were isolated from the blood of healthy anonymous donors (Bloodworks Northwest) using HetaSep™ (catalog number 07906, StemCell Technologies) following the manufacturer's instructions. Neutrophils were identified as CD45, CD11b and CD66b positive cells as assessed by flow cytometry.
(7) Gene Editing. To delete Siglec-9 expression, LAD2 cells were transfected with Siglec-9-targeting ribonucleoproteins using Lipofectamine CRISPRMAX reagent (catalog number CMAX00001, Thermo Fisher Scientific). Transfection was performed based on the manufacturer recommendations as follows. Cells were plated at 3×10⁵ cells/well of a 24-well dish and transfected with equimolar (12 pmol) Cas9 (catalog number 1081058, Integrated DNA Technologies, Coralville, IA) and sgRNA (5′-GACGAUGCAGAGUUCCGUGA-3′ (SEQ ID NO: 23)) (catalog number A35533, Thermo Fisher Scientific) complexed with 4 μl Cas9Plus reagent and 1.5 μl CRISPRMAX reagent according to the manufacturer's mixing instructions. Editing efficiency was evaluated by protein expression measured by flow cytometry.
(8) qPCR. RNA was isolated from primary human mast cell using the RNeasy Plus Micro kit (catalog number 74034, Qiagen, Hilden, Germany). RNA was isolated from mast cell lines using the RNeasy Mini kit (catalog number 74104, Qiagen), converted to first-strand cDNA (SuperScript™ VILO™ CDNA Synthesis Kit, Invitrogen), and cDNA was analyzed for quantitative expression levels with the Maxima SYBR Green/ROX qPCR Master Mix (Thermo Fisher Scientific) on a Step One Plus Real-Time PCR System Thermal Cycling Block (Applied Biosystems, Waltham, MA). Results were analyzed using the dCt method normalized to GAPDH Ct. The primers used were as follows:

SIGLEC7 forward,
(SEQ ID NO: 3)
5′-GCCATAAGTTTGCAGCATCTC-3′;
SIGLEC7 reverse,
(SEQ ID NO: 4)
5′-GCCATTGGAAGCTCTATCTGC-3′;
SIGLEC8 forward,
(SEQ ID NO: 5)
5′-GTTGGGGGTGAAGTCAGAAAAG-3′;
SIGLEC8 reverse,
(SEQ ID NO: 6)
5′-GGGTGGGAATCTGGATGAGTT-3′;
SIGLEC9 forward,
(SEQ ID NO: 7)
5′-AATCTGACCTGCTCTGTGCC-3′;
SIGLEC9 reverse,
(SEQ ID NO: 8)
5′-AAGTTCTGAGGCGGGTAGGA-3′;
ST3GAL1 forward,
(SEQ ID NO: 9)
5′-CCGCTGTGGTCATTTAGGAA-3′;
ST3GAL1 reverse,
(SEQ ID NO: 10)
5′-TCCATCTCTGGTCCCCAAAT-3′;
ST3GAL3 forward,
(SEQ ID NO: 11)
5′-CCGCTGTGGTCATTTAGGAA-3′;
ST3GAL3 reverse,
(SEQ ID NO: 12)
5′-GGGGTGAGCTAGAGTGACTA-3′;
ST3GAL4 forward,
(SEQ ID NO: 13)
5′-CTCCCGGGAAGACAGTTTTT-3′;
ST3GAL4 reverse,
(SEQ ID NO: 14)
5′-GTAAGCAGATGGCGTCTTGA-3′;
ST3GAL5 forward,
(SEQ ID NO: 15)
5′-CTCTGTGGCTGCTCTTGTCA-3′;
ST3GAL5 reverse,
(SEQ ID NO: 16)
5′-TGGTGAGGAGGAGGGAGATG-3′;
ST3GAL6 forward,
(SEQ ID NO: 17)
5′-CTGCTCCCTCCTCTGCTC-3′;
ST3GAL6 reverse,
(SEQ ID NO: 18)
5′-CCGGCAGAAACCAAAGGAA-3′;
GNE forward,
(SEQ ID NO: 19)
5′-TGACGGCGTCTGGAACTCTA-3′;
GNE reverse,
(SEQ ID NO: 20)
5′-GTAGCAACACAAACCCGCAG-3′;
GAPDH forward,
(SEQ ID NO: 21)
5′-GAGTCAACGGATTTGGTCGT-3′;
GAPDH reverse,
(SEQ ID NO: 22)
5′-TTGATTTTGGAGGGATCTCG-3′.

(9) Flow cytometry. Single human cell suspensions were stained with a combination of the following antibodies: Alexa Fluor-700-conjugated anti-human Siglec-6 (clone 767329, catalog number FAB2859N-025, R&D Systems, Minneapolis, MN); PE-conjugated anti-human Siglec-7 (clone 194211, catalog number FAB11381P, R&D Systems); BV421-conjugated anti-human Siglec-8 (clone 837535, catalog number 747875, BD Biosciences, Franklin Lakes, NJ); BV421-conjugated anti-human Siglec-9 (clone E10-286, catalog number 743363, BD Biosciences); APC-conjugated anti-human Siglec-9 (clone 191240, catalog number FAB1139A, R&D Systems); BV605-conjugated anti-human c-Kit (clone 104D2, catalog number 562687, BD Biosciences); FITC-conjugated anti-human FcεRIα (clone CRA-1, catalog number 11-5899-42, Invitrogen, Waltham, MA); APC/Cy7-conjugated anti-human lysosomal-associated membrane protein 1 (LAMP-1) (clone H4A3; catalog number 328629, Biolegend); FITC-conjugated anti-human CD45 (clone 2D1, catalog number 11-9459-41, Invitrogen); APC/Cy7-conjugated anti-human CD11b (clone ICRF44, catalog number 301341, Biolegend); BV421-conjugated anti-human CD66b (clone 6/40c, catalog number 392915, Biolegend).

Single mouse cell suspensions were stained with a combination of the following antibodies: anti-APC/Cy7-conjugated anti-mouse c-Kit (clone 2B8, catalog number 105825, Biolegend); PE-conjugated anti-mouse FcεRIα (clone MAR-1, catalog number 134307, Biolegend); and BV421-conjugated anti-mouse Siglec-E (clone 750620, catalog number 748154, BD Biosciences). 4,6-Diamidino-2-phenylindole (DAPI) (catalog number D9542, Sigma Aldrich) was used to exclude dead cells.

For Siglec-9 and LAMP-1 staining in PBCMCs and HSCMCs, cells were untreated or stimulated with 100 ng/ml anti-human FcεRIα (clone CRA-1, catalog number 14-5899-82, Invitrogen) for 30 min at 37° C.

In a separate group of experiments, LAD2 cells and HSCMCs were incubated with 10 mU/ml sialidases (catalog number 11080725001, Roche, Indianapolis, IN) for 1 h at 37° C. as previously described (Liu et al., J Virol, 72:4643-9, 1998) followed by LAMP-1 staining.

Receptor endocytosis was determined by delayed secondary staining as previously described (O'Sullivan et al., J Allergy Clin Immunol, 141:1774-85 e7, 2018). Briefly, Siglec-9 was bound with 5 μg/ml unconjugated mouse anti-human Siglec-9 (clone 191240, R&D Systems) or mouse IgG2a isotype control antibody (catalog number MAB003, R&D Systems) for 20 min on ice followed by incubation at 37° C. for 1, 24, or 48 h. At indicated time points, 1:1000 dilution of an Alexa Fluor-594-conjugated donkey anti-mouse IgG antibody (catalog number A21203, Invitrogen) was added to assess the remaining Siglec-9 on the cell surface by flow cytometry. Initial levels of antibody binding were determined on cells incubated at 4° C. after ligation of Siglec-9. To assess total Siglec-9 levels, cells were stained with APC-conjugated anti-Siglec-9. The percentage of signal lost was calculated as follows: 100×(1-mean intensity fluorescence (MFI) at a specific time/MFI at time 0).

To determine expression of Siglec-9 ligands, untreated or sialidase-treated mast cells were incubated with 10 μg/ml rhSiglec-9 Fc chimera protein (catalog number 1139-SL-050, R&D Systems) for 30 min on ice followed by incubation with a 1:200 dilution of an Alexa Fluor 488-conjugated anti-human IgG secondary antibody (catalog number 709-546-149, Jackson ImmunoResearch, West Grove, PA) for 30 min on ice. Baseline staining was obtained using PBS followed by the fluorescently labeled secondary antibody.

Mast cell apoptosis was determined in cells cultured with 5 μg/ml mouse anti-human Siglec-9 (clone 191240) or mouse IgG2a isotype control for 20 min on ice followed by washing and incubation at 37° C. in warmed media for 1, 24, or 48 h. At indicated time points, cells were collected, stained with propidium iodide (catalog number 421301, Biolegend) and FITC-conjugated Annexin V (catalog number 640905, Biolegend) in Annexin V binding buffer (catalog number 422201, Biolegend), and analyzed by flow cytometry.

Data was acquired on a BD LSR II with the FACSDIVA software and analyzed using FlowJo software (for Windows, version 10, Becton, Dickinson, and Company, Ashland, OR).
(10) Microscopy. For Siglec-9 imaging, cells were plated on chamber slides (catalog number 154526, Thermo Fisher Scientific) coated with 40 μg/ml human plasma fibronectin (catalog number F2006, Sigma Aldrich) for 1 h at 37° C. Cells were fixed with 3% paraformaldehyde for 15 min at room temperature, washed 3 times with PBS, permeabilized with 0.5% Triton X-100 for 10 min, and blocked with 5% normal goat serum for 1 h at room temperature. Cells were then incubated overnight with mouse anti-human Siglec-9 (clone 191240) at 4° C., washed 3 times with PBS, incubated with 1:200, Alexa Fluor 594-conjugated donkey anti-mouse IgG for 30 min at room temperature, and washed 3 times with PBS. Samples were mounted using Prolong Diamond DAPI containing mounting media (catalog number P36966, Invitrogen). Images were acquired on a Leica TCS SP5 confocal inverted-base microscope (Leica Microsystems, IL) with a 63× oil objective and analyzed using ImageJ (NIH).

For analysis of endocytosis upon Siglec-9/FcεRI co-crosslinking, cells were fixed, permeabilized, and stained as above, but in suspension rather than adhered to slides and finally resuspended in SlowFade DAPI-containing mounting media (catalog number S36968, Invitrogen) before imaging. Endocytosis analysis was performed with the following additional antibodies: rabbit anti-Rab5a (catalog number 2143S, Cell Signaling Technology, Danvers, MA), rabbit anti-Rab7 (clone D95F2, catalog number 9367S, Cell Signaling Technology), Alexa Fluor 594-conjugated donkey anti-rabbit IgG (catalog number ab150076, Abcam, Cambridge, UK), and Alexa Fluor 488-conjugated donkey anti-mouse IgG (A21202, Invitrogen).

In a separate experiment, cells tested for Siglec-9 ligand expression by flow cytometry were attached to a slide by cytospin (500×g, 5 min), fixed using 4% paraformaldehyde for 10 min, washed with PBS, and imaged under oil immersion at 63×using a Leica DM6000 (Leica, Wetzlar, Germany) microscope.
(11) Siglec-9 engagement with native ligands. Mock- and SIGLEC9-edited LAD2 cells sensitized overnight with 2 μg/ml of human IgE (catalog number ab65866, Abcam) and HSCMCs were cultured with either 20-100 μg/ml glycophorin A (GlycA) (catalog number G5017, Sigma Aldrich) or 20-100 μg/mL high molecular weight hyaluronic acid (HMW-HA) (catalog number 53747, Sigma Aldrich) for 20 min. The cells were then activated with the indicated concentrations of stimulants for 30 min at 37° C. LAD2 cell and HSCMC activation was assessed by β-hexosaminidase release and increase in LAMP-1 expression, respectively. Percentage of inhibition of mast cell degranulation was calculated as follows: (% of β-hexosaminidase release or LAMP-1 expression in stimulated cells-β-hexosaminidase release or LAMP-1 expression in untreated cells)-(% of β-hexosaminidase release or LAMP-1 expression in stimulated cells treated with Siglec-9 ligand-β-hexosaminidase release or LAMP-1 expression in untreated cells)/(% of β-hexosaminidase release or LAMP-1 expression in stimulated cells-β-hexosaminidase release or LAMP-1 expression in untreated cells).
(12) Siglec-9 engagement with anti-Siglec-9 antibody. Mast cells were pre-incubated with 2.5-5 μg/ml mouse anti-Siglec-9 (clone 191240) or mouse IgG2a isotype control antibody for 30 min at room temperature prior to activation with the indicated concentrations of stimulants for 30 min at 37° C.

In a separate set of experiments, PBCMCs incubated with anti-Siglec-9 or isotype control were exposed to 5 μg/ml goat anti-mouse IgG (Fc specific) F(ab′)2 fragment antibody (catalog number M0284, Sigma-Aldrich) for 2 min to cross-link Siglec-9 prior to activation.

LAD2 cell activation was assessed by β-hexosaminidase release, and PBCMC and HSCMC activation was assessed by LAMP-1 staining.
(13) Co-crosslinking of FcεRI and Siglec-9. PBCMCs and HSCMCs were incubated with 100 ng/ml anti-human FcεRIα plus 5 μg/ml mouse isotype control antibody (catalog number MAB003, R&D Systems) or anti-Siglec-9 mAb (clone 191240) for 2 min at 4° C. Cells were washed and then incubated with 5 μg/ml goat anti-mouse IgG (Fc specific) F(ab′)2 fragment antibody for 2 min. After an additional PBS wash, cells were resuspended in warm complete medium and incubated for 30 min at 37° C. to assess mast cell activation by LAMP-1 staining. Cell supernatants were collected at 20 min and after overnight stimulation to measure the levels of arachidonic acid metabolites, and chemokine/cytokines, respectively.
(14) β-hexosaminidase release assay. LAD2 cells were sensitized with 2 μg/ml of human IgE by overnight incubation at 37° C. The cells were then washed with Tyrode's buffer (10 mM HEPES, pH 7.4, 130 mM NaCl, 5 mM KCl, 1.4 mM CaCl₂), 1 mM MgCl₂, and 0.1% glucose) and 1×10⁵ cells/well were added to a 96-well V-bottom plate. Next, equal volume of a 2× concentration of stimulus (500 ng/ml anti-human IgE (clone B3102E8, catalog number ab99804, Abcam), 10 μM compound 48/80 (c48/80) (catalog number C2313, Sigma Aldrich) or 1 μM A23187 (catalog number C7522, Sigma Aldrich) was added to the appropriate wells, and incubated at 37° C. for 1 h. After centrifugation, supernatants were collected, and pellets were lysed with 0.5% Triton X 100. β-hexosaminidase release was quantified by enzyme immunoassay with p-nitrophenyl-N-acetyl-β-d-glucosamine (catalog number N9376, Sigma-Aldrich) substrate, as follows: 10 μl of culture supernatant or lysate was added to the wells of a 96-well flat-bottom plate; 50 μl of 1.3 mg/ml p-nitrophenyl-N-acetyl-β-d-glucosamine solution in 100 mM sodium citrate, pH 4.5, was added, and the plate was incubated at 37 C for 1 h. Next, 150 μl of 200 mM glycine, pH 10.7, was added to stop the reaction, and the optical density (OD₄₀₅) was determined.
(15) Prostaglandin (PG) D2 and cysteinyl leukotriene (cys-LT) release assays. PGD₂ and cys-LT levels were measured in supernatants by ELISA per the manufacturer's instructions (catalog numbers 512031 and 500390 for PGD₂ and cys-LT, respectively, Cayman Chemical, Ann Arbor, MI). The assay detection limits were 19.5 pg/ml and 8.6 pg/ml for PGD₂ and cys-LT, respectively.
(16) Chemokine release assay. IL-8 and monocyte chemoattractant protein (MCP)-1 levels were measured in cell supernatants using the human ProcartaPlex Multiplex ImmunoAssay (catalog number PPX-07-MXH6CA4, Thermo Fisher Scientific). The assay detection limits were 2.49 pg/ml for IL-8 and 4.16 pg/ml for monocyte chemoattractant protein-1 (MCP-1).
(17) Statistical Analyses. Data are presented as means±SEMs unless otherwise indicated. Statistical significance was determined by Mann Whitney U test, Wilcoxon matched pair signed rank test, or ANOVA and Tukey multiple comparisons test as appropriate, using GraphPad Prism version 9 (San Diego, CA, USA). When appropriate, single outliers were identified and removed from the data using the ROUT outlier test. Statistical differences were considered significant at P<0.05.

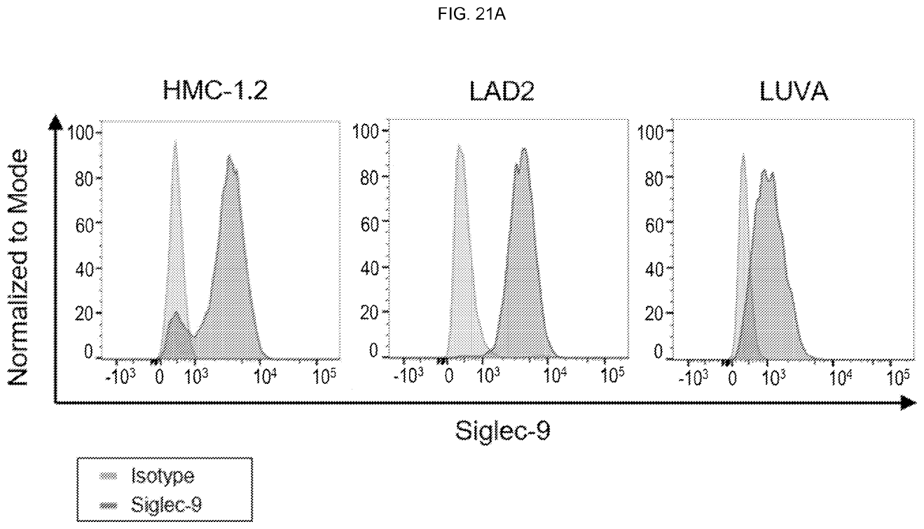

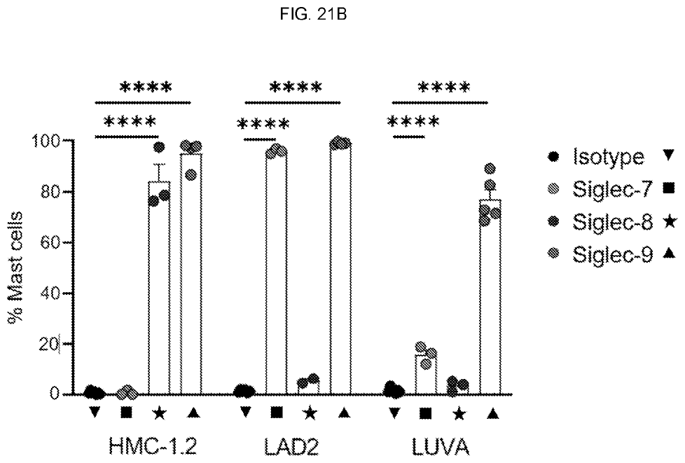

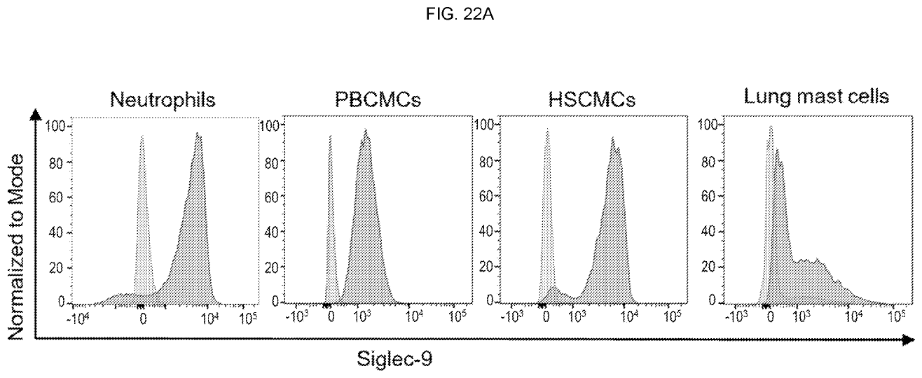

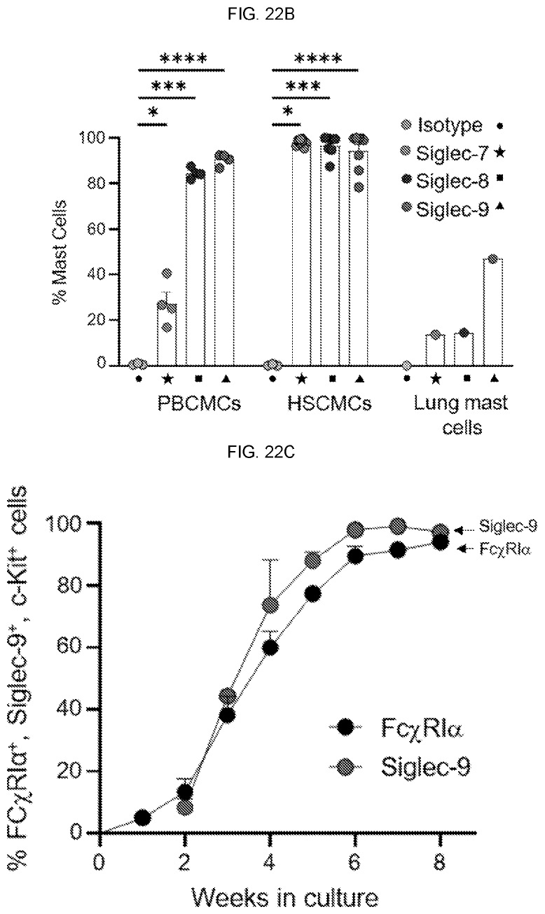

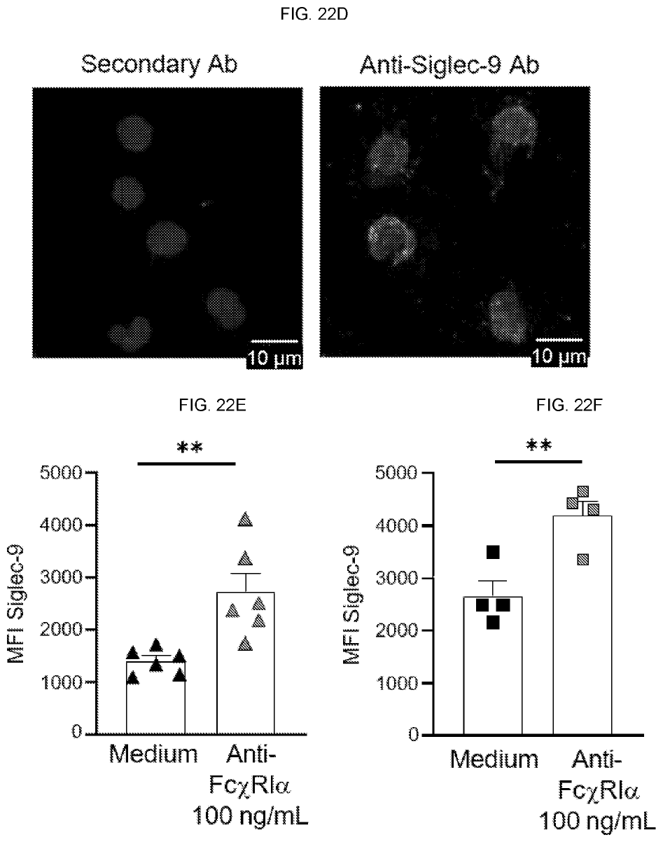

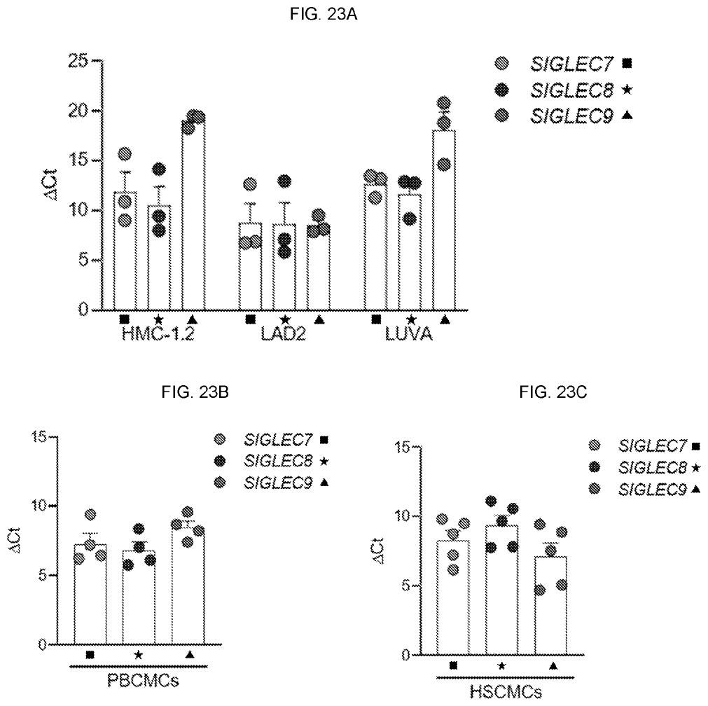

(D) Results. (1) Human mast cells express Siglec-9. First, the surface level expression of Siglec-9 was compared across a variety of human mast cell types. It was found that human mast cell lines (FIG. 21A), PBCMCs, HSCMCs, and lung mast cells express Siglec-9 (FIG. 22A). Human neutrophils isolated from peripheral blood were used as a positive control for Siglec-9 protein expression (FIG. 22A). When compared to other Siglecs, human primary mast cells expressed Siglec-9 levels comparable to Siglec-7 and Siglec-8 (FIG. 22B). Moreover, Siglec-9 was the only Siglec expressed by all the human mast cell lines tested (FIG. 21B). At the mRNA level, human mast cell lines, PBCMCs, and HSCMCs expressed SIGLEC9 mRNA levels comparable to Siglec-7 and Siglec-8 (FIGS. 23A-23C).

As previously reported for Siglec-6 and Siglec-8 (Yokoi et al., Allergy, 61:769-76, 2006), kinetics studies showed that Siglec-9 appeared on the cell surface in parallel with FcεRIα expression (FIG. 22C), indicating that Siglec-9 expression increases with mast cell maturation.

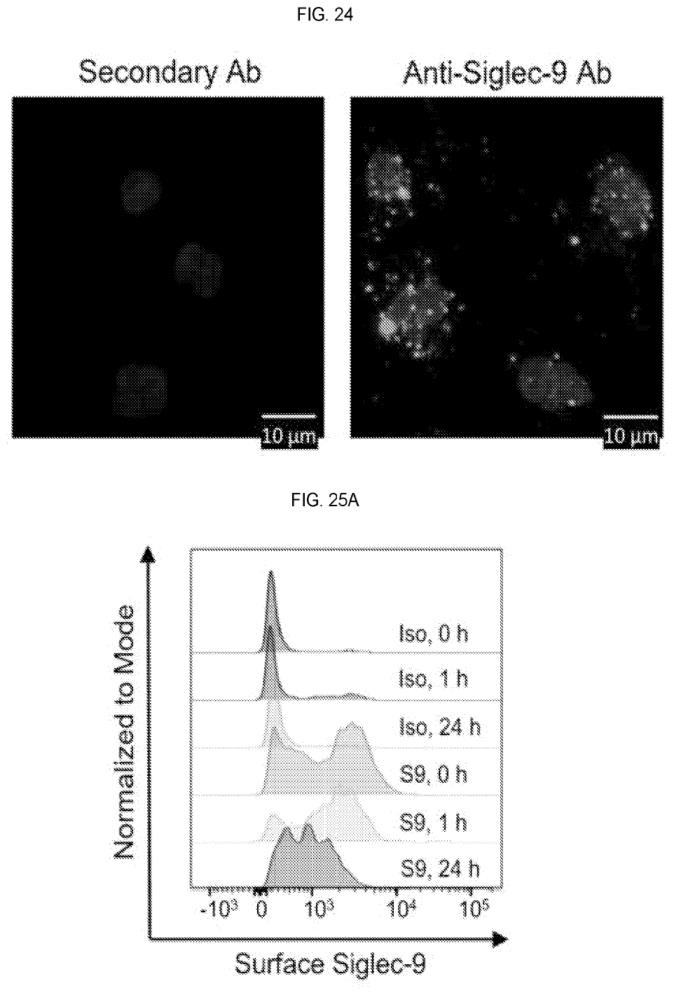

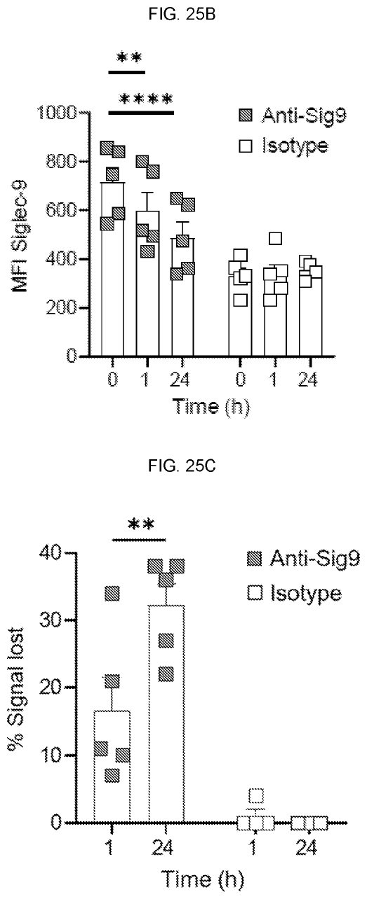

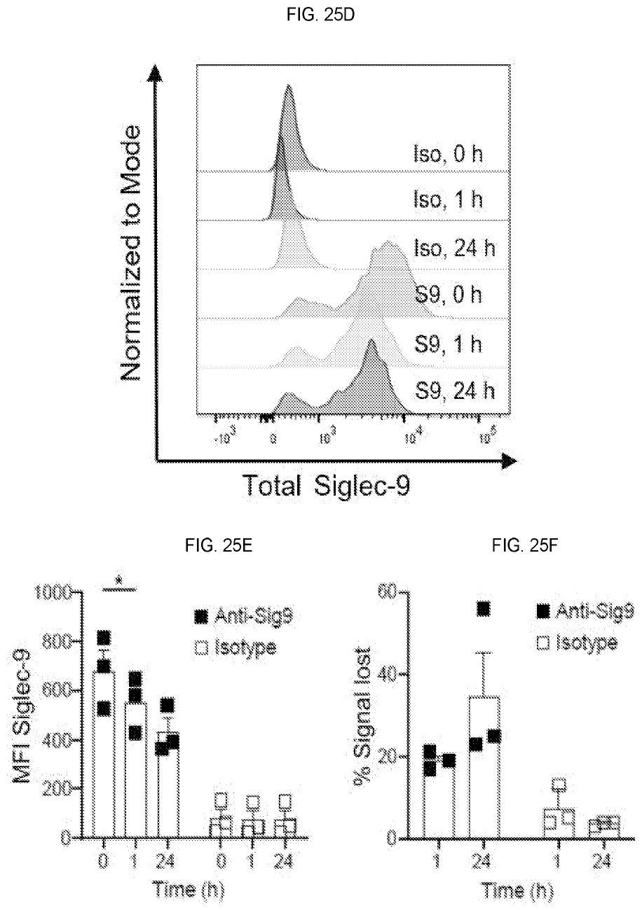

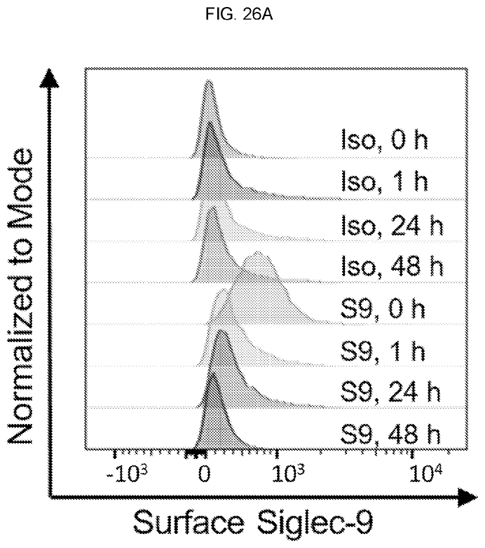

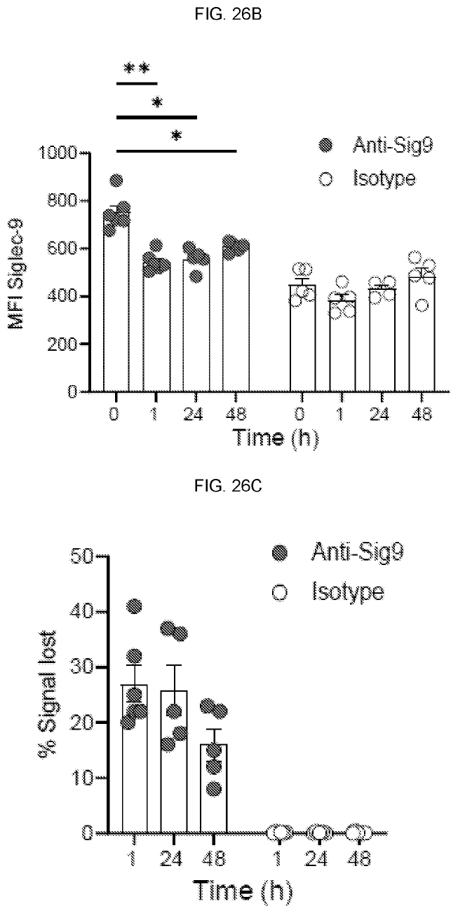

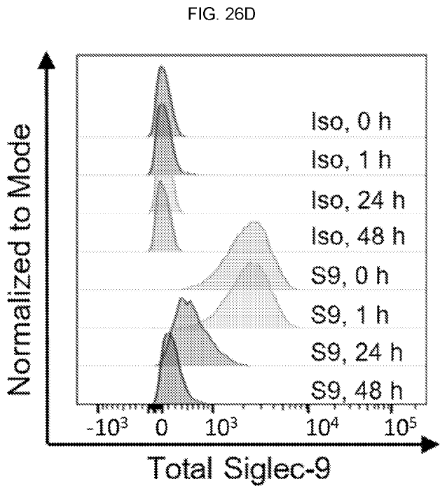

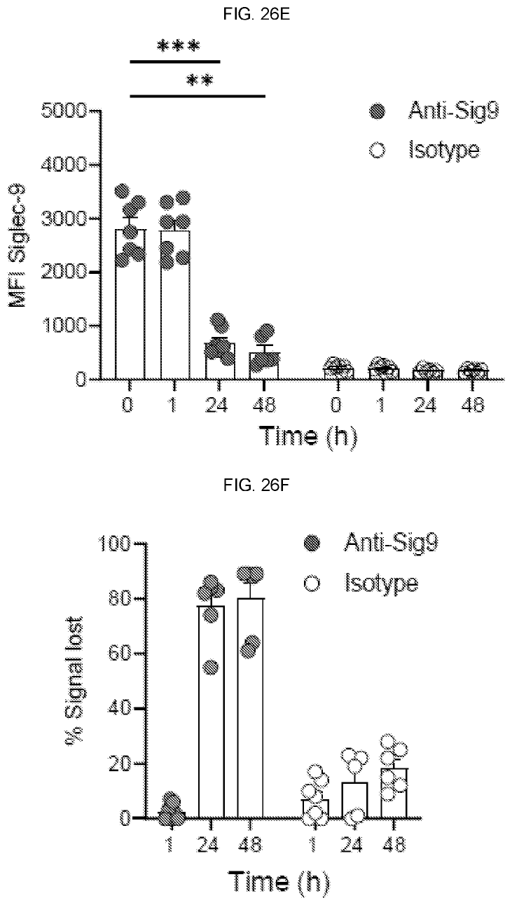

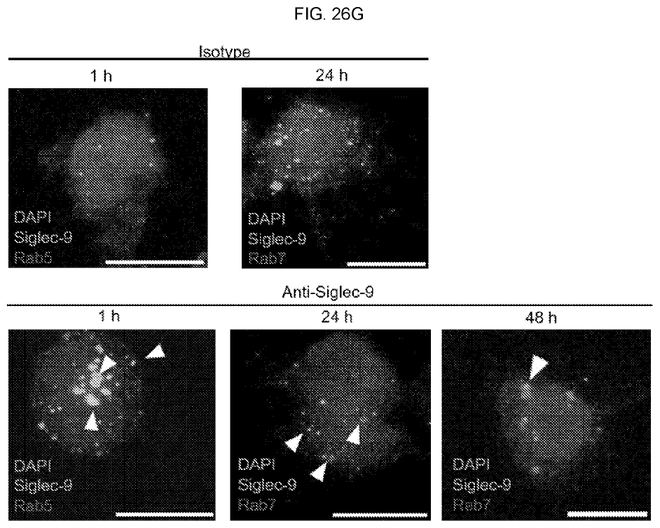

Confocal microscopy for Siglec-9 localization in LAD2 cells (FIG. 24) and PBCMCs (FIG. 22D) showed a punctate distribution of Siglec-9, which has previously been described in other cell types (Avril et al., J Immunol, 173:6841-9, 2004; Laubli et al., Proc Natl Acad Sci USA, 111:14211-6, 2014; Ikehara et al., J Biol Chem, 279:43117-25, 2004). Given the punctate intracellular staining of Siglec-9 in PBCMCs, it was hypothesized that there is a potential intracellular storage of Siglec-9. To test this, PBCMCs and HSCMCs were stimulated in an IgE-dependent manner and assessed for surface expression of Siglec-9. Both PBCMCs (FIG. 22E) and HSCMCs (FIG. 22F) showed a significant increase in Siglec-9 expression after undergoing IgE-dependent degranulation, suggesting that Siglec-9 can be incorporated into the cell membrane after degranulation. Together, these data demonstrate that human mast cells express Siglec-9 on their cell surface and intracellularly.
(2) Siglec-9 is an endocytic receptor. Siglec-6 and Siglec-8 are internalized after antibody engagement of the receptor on eosinophils and mast cells (O'Sullivan et al., J Leukoc Biol, 108:73-81, 2020; Robida et al., Cells, 11, 2022). The endocytic capacity of Siglec-9 has only been described in cancer cells (Biedermann et al., Leuk Res, 31:211-20, 2007); where up to 90% of Siglec-9 was internalized after treatment with anti-Siglec-9 antibodies. Therefore, it was first tested whether Siglec-9 ligation causes a decrease in Siglec-9 surface expression using the previously described delayed secondary staining method (O'Sullivan et al., J Leukoc Biol, 108:73-81, 2020). After ligation of Siglec-9 with an anti-Siglec-9 monoclonal antibody (clone 191240), there was a gradual decrease in surface Siglec-9 staining on HSCMCs (FIGS. 25A and 25B). As shown in FIG. 25C, 30% of the Siglec-9 signal was lost at 24 h after antibody engagement when compared with Siglec-9 signal detected at time 0. In contrast, isotype control treated cells did not exhibit a decrease in Siglec-9 surface expression at any timepoint (FIG. 25A-25C). Siglec-9 surface expression decreased more rapidly in LAD2 cells (FIG. 26A, 26B), which exhibited a 30% loss in Siglec-9 surface expression at 1 h after Siglec-9 ligation (FIG. 26C). The MFI slightly increased and the percentage in surface Siglec-9 loss decreased at 24 h after Siglec-9 ligation, suggesting that a small portion of the original Siglec-9 started returning to the LAD2 cell surface (FIGS. 26B and 26C). In contrast, LAD2 cells incubated with isotype control did not exhibit any loss in Siglec-9 surface expression (FIGS. 26A-26C). Together, this evidence shows that Siglec-9 surface expression decreases after receptor engagement.

To further characterize whether the Siglec-9 loss was due to endocytosis, the expression of surface and internalized Siglec-9 (Total Siglec-9) was assessed by using a fluorophore-conjugated anti-Siglec-9 antibody. HSCMCs exhibited a significant loss in total Siglec-9 signal at 1 h after antibody engagement (FIG. 25D). Isotype treated cells showed no Siglec-9 signal during the times tested (FIGS. 25D and 25E). Loss in total Siglec-9 signal was more pronounced in LAD2 cells which exhibited an almost complete loss in total Siglec-9 signal at 24 h after Siglec-9 ligation (FIGS. 26D-26F). Based on this data, it was hypothesized that Siglec-9 was degraded after endocytosis. To address this hypothesis, whether Siglec-9 co-localizes with markers of the endosomal pathway in LAD2 cells after Siglec-9 engagement was examined. Confocal images showed that Siglec-9 co-localized with Rab5, a marker of early endosomes, and with Rab7, a marker of late endosomes, at 1 h and 24 h after Siglec-9 engagement, respectively (FIG. 26G). In contrast, Siglec-9 did not co-localize with Rab5 or Rab7 in LAD2 cells treated with isotype control at any timepoint tested (FIG. 26G).

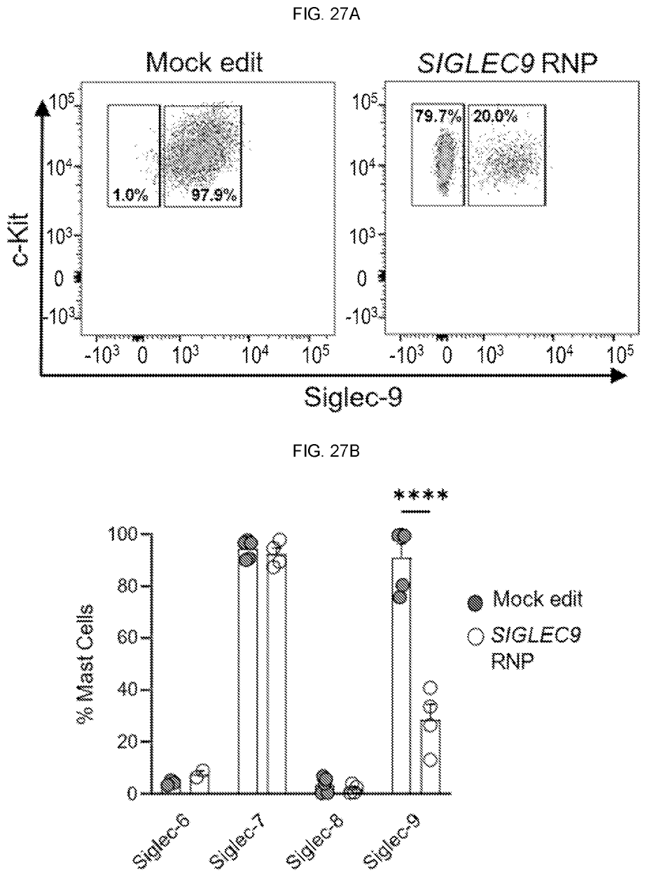

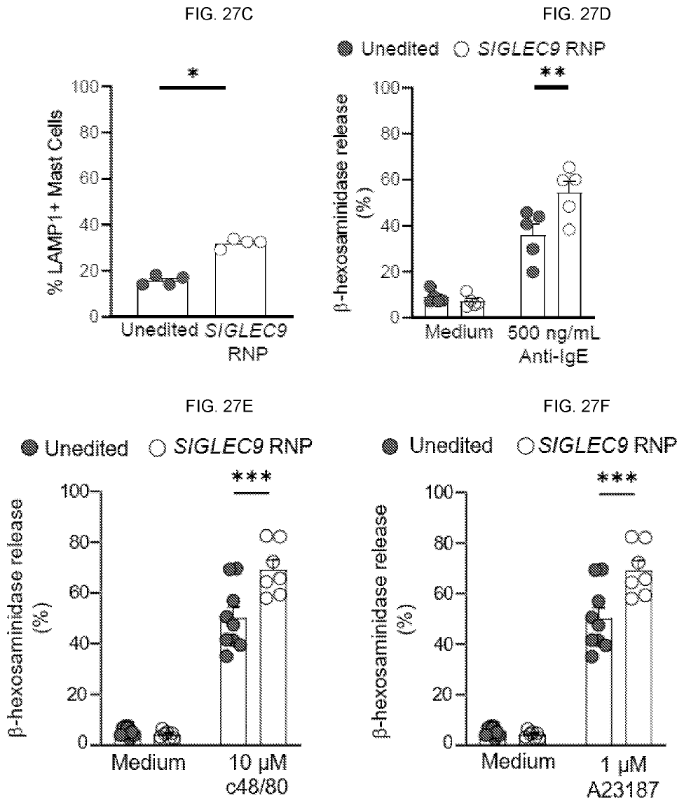

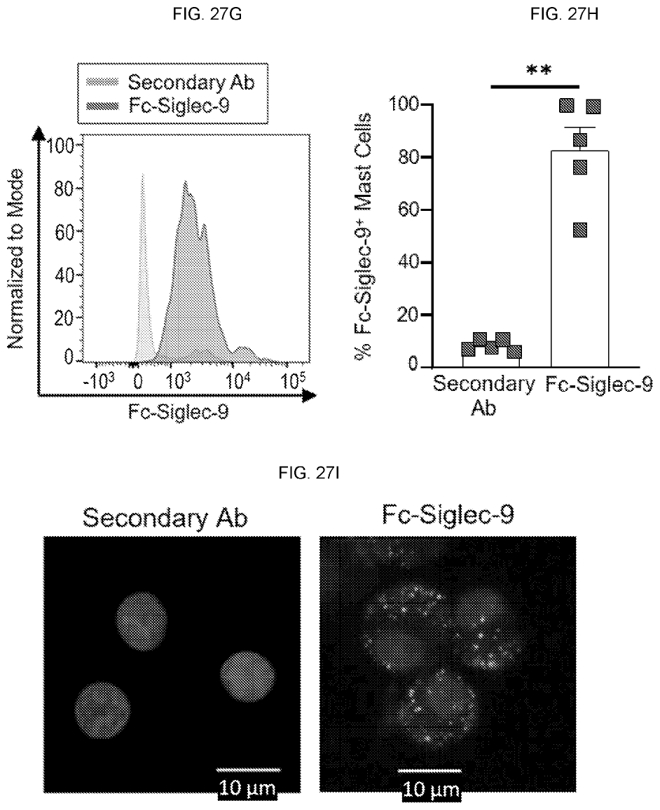

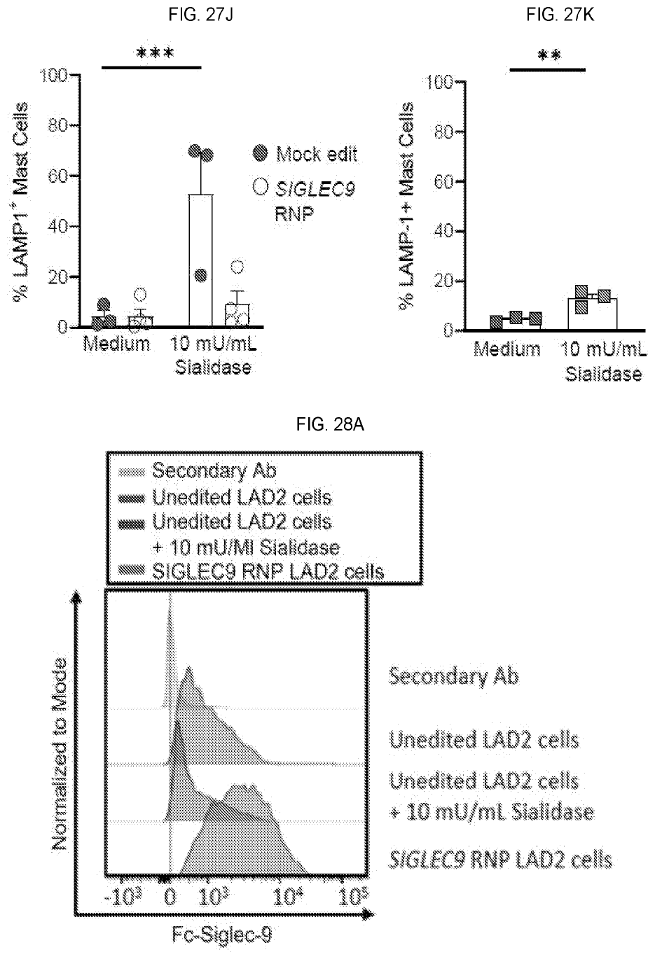

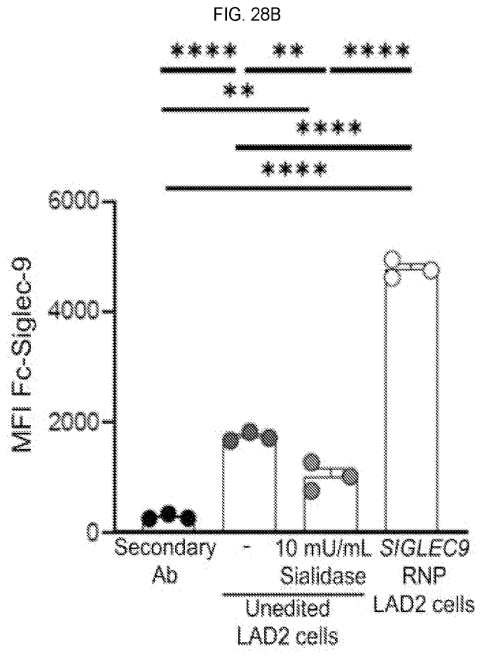

Together, this data suggests that Siglec-9 is internalized and degraded upon engagement with anti-Siglec-9 antibodies.
(3) Siglec-9 interactions with sialic acids in cis dampen mast cell susceptibility to activation. Next, a human mast cell line deficient in Siglec-9 was generated to assess the role of Siglec-9 in mast cell function. For this purpose, Siglec-9 was deleted in LAD2 cells using a CRISPR/Cas9 approach. By using a CRISPR/Cas9 approach, a large population of LAD2 cells that do not express Siglec-9 was generated (FIG. 27A). In contrast, Siglec-6, Siglec-7, and Siglec-8 expression levels were unaffected by Siglec-9 deletion in LAD2 cells (FIG. 27B). First, it was observed that naïve Siglec-9-deleted LAD2 cells exhibited increased cell surface expression levels of LAMP-1 which indicates granule mobilization towards the plasma membrane (FIG. 27C). Moreover, Siglec-9-deleted LAD2 cells were more susceptible to degranulation induced by IgE-dependent (FIG. 27D) and IgE-independent stimuli (FIGS. 27E and 27F). These observations support the presence of sialic acid ligands with the ability to trigger an inhibitory signal in mast cells via Siglec-9 engagement. To address this hypothesis, mast cells were probed for the presence of Siglec-9 ligands using a recombinant chimera of Siglec-9 (Fc-Siglec-9) as previously described (Ibarlucea-Benitez et al., Proc Natl Acad Sci USA, 118, 2021). HSCMCs showed expression of Siglec-9 ligands by flow cytometry (FIGS. 27G and 27H). Imaging of the Siglec-9 ligand expression in HSCMCs showed a punctate pattern of expression, suggesting there is an enrichment of these ligands in certain areas of the cell membrane (FIG. 27I). As shown in FIGS. 28A and 28B, LAD2 cells also express significant levels of Siglec-9 ligands when compared with cells treated with secondary antibody alone. The staining for Siglec-9 ligands in LAD2 cells was abrogated when cells were treated with sialidases to remove potential Siglec ligands, demonstrating that Fc-Siglec-9 chimeras specifically bind to sialic acid residues on the cell membrane (FIGS. 28A and 28B). Notably, Siglec-9 deleted LAD2 cells showed significantly higher staining for Siglec-9 ligands (FIGS. 28A and 28B) suggesting that binding of Siglec-9 to sialic acids may interfere with Siglec-9 ligand detection by Fc-Siglec-9 chimeras in unedited LAD2 cells.

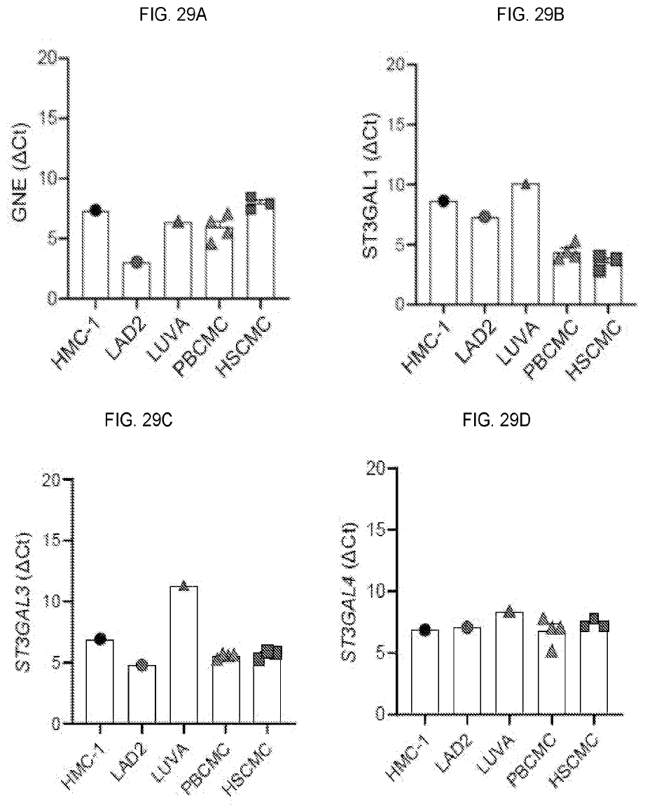

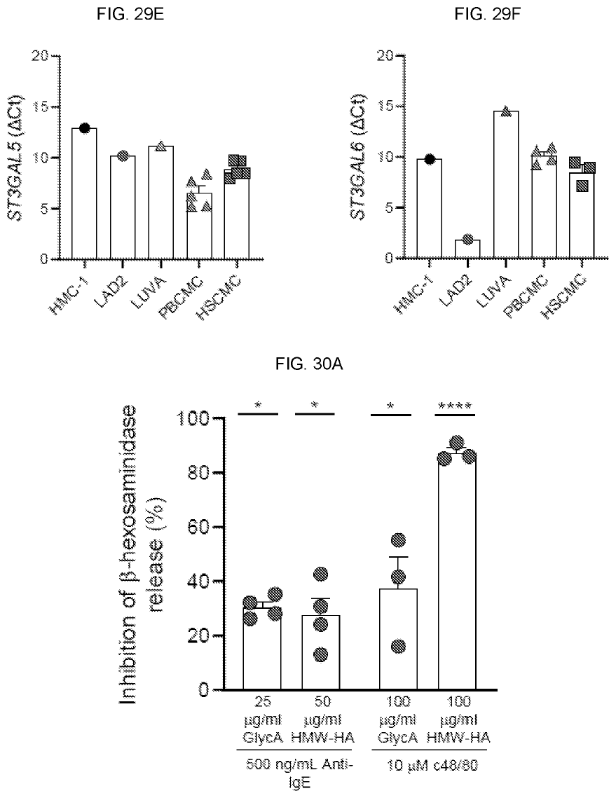

These findings prompted the exploration of whether mast cells express enzymes involved in sialic acid biosynthesis. Sialic acid biosynthesis starts with the formation of cytidine monophosphate N-acetylneuraminic acid (CMP-Neu5Ac) (Hugonnet et al., Front Immunol, 12:799861, 2021) as an activated sugar donor for the transfer of sialic acids by sialyltransferases (SiaT) to the terminal glycosyl group of glycoproteins and glycolipids as acceptor molecules. As shown in FIGS. 29A-29F, it was found that human mast cells express mRNA for UDP-GIcNAc 2-epimerase/ManNAc-6 (GNE), the first enzyme in the pathway to generating CMP-Neu5Ac (Stasche et al., J Biol Chem, 272:24319-24, 1997). Moreover, human mast cells express mRNA for SiaTs that can catalyze the formation of glycosidic linkages found in Siglec-9 ligands including ST3GAL1, ST3GA3, ST3GAL4, ST3GAL5 and ST3GAL6. Overall, this evidence supports the expression and biosynthesis of Siglec-9 ligands in human mast cells.

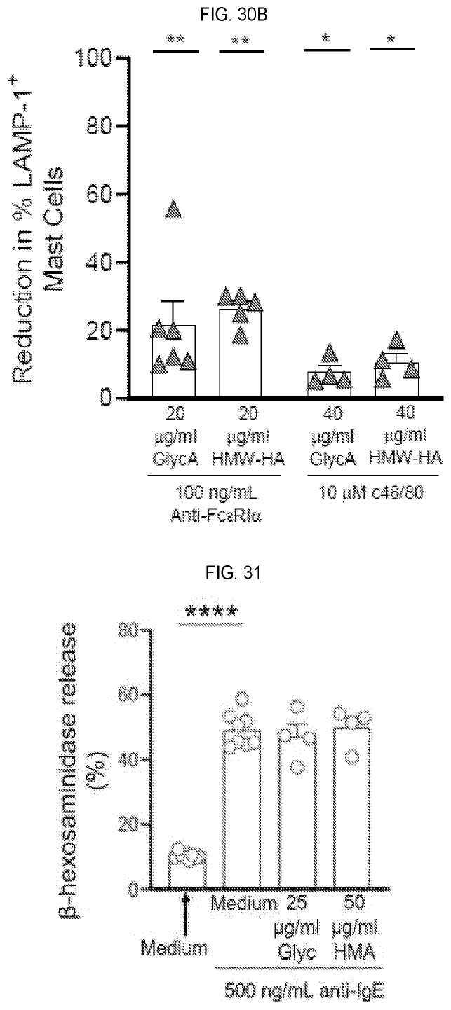

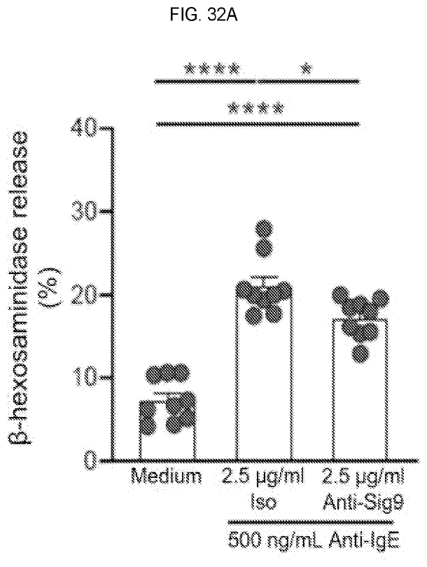

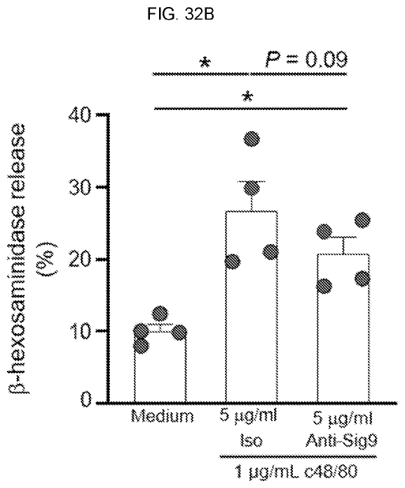

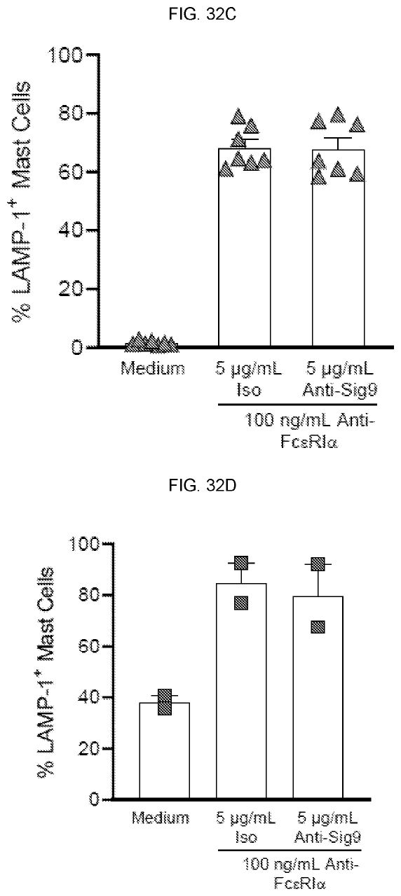

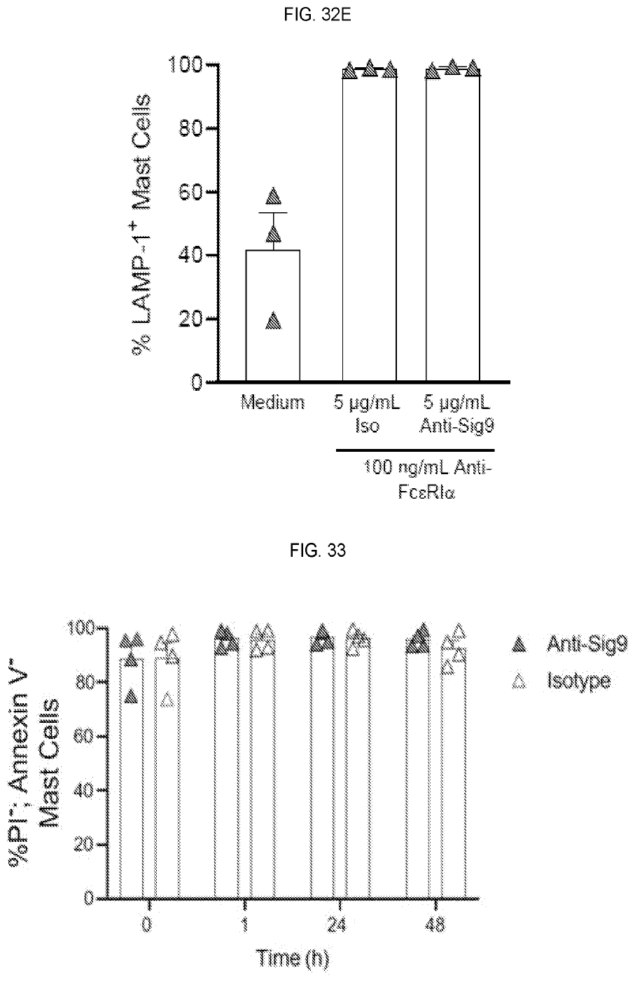

To address whether the presence of these sialic acid ligands can trigger an inhibitory signal in mast cells via Siglec-9 engagement, LAMP-1 expression in mast cells devoid of potential Siglec ligands was examined. As shown in FIG. 27J, unedited but not Siglec-9-deleted LAD2 cells exhibited increased LAMP-1 expression after sialidase treatment. Increased LAMP-1 expression was also observed when HSCMCs were treated with sialidases, indicating that sialic acid ligands also limit the extent of human primary mast cell activation (FIG. 27K). Together, these data show that mast cells express Siglec-9 ligands that can engage Siglec-9 to promote mast cell quiescence.
(4) Siglec-9 ligands reduce mast cell activation. GlycA and HMW-HA have been recognized as ligands for Siglec-9 that can downregulate the activation of immune cells (Secundino et al., J Mol Med (Berl), 94:219-33, 2016; Lizcano et al., Blood, 129:3100-10, 2017). Therefore, the effects of GlycA and HMW-HA on mast cell activation were assessed. As shown in FIG. 30A, optimal concentrations of both ligands inhibited β-hexosaminidase release from LAD2 cells stimulated by IgE-dependent and IgE-independent stimuli. In contrast, Siglec-9 deleted LAD2 cells did not exhibit a reduction in their ability to release β-hexosaminidase when incubated with Siglec-9 ligands suggesting that these ligands inhibit mast cell activation by specifically engaging Siglec-9 (FIG. 31). Both GlycA and HMW-HA were able to inhibit PBCMC degranulation as assessed by a decrease LAMP-1 expression levels upon stimulation by IgE-dependent (FIG. 30B). In contrast, Siglec-9 ligands did not inhibit PBCMC degranulation induced by IgE-independent stimuli (FIG. 30B). These results point to naturally occurring Siglec-9 ligands like GlycA and HMW-HA being able to further dampen mast cell activation through their engagement of Siglec-9.
(5) Antibody engagement of Siglec-9 inhibits mast cell activation. Antibodies against Siglecs have been used successfully to engage these inhibitory receptors and modulate immune and inflammatory responses (Landolina et al., Pharmacol Res, 158:104682, 2020; Schanin et al., Mucosal Immunol, 14:366-76, 2021; Youngblood et al., JCI Insight, 4, 2019; Kerr et al., Clin Exp Allergy, 50:904-14, 2020; Gebremeskel et al., Front Immunol, 12:650331, 2021). To determine whether antibody engagement of Siglec-9 reduces mast cell activation, LAD2 cells were incubated with an anti-Siglec-9 monoclonal antibody (clone 191240) for 30 min prior to mast cell stimulation. Siglec-9 antibody engagement inhibited mast cell degranulation induced by IgE-dependent (FIG. 32A) and IgE-independent stimuli (FIG. 32B) in LAD2 cells. In contrast, PBCMCs and HSCMC did not exhibit a reduction in their ability to degranulate when treated with anti-Siglec-9 antibodies (FIGS. 32C and 32D). PBCMC degranulation was also not inhibited when anti-Siglec-9 antibodies were crosslinked with a secondary antibody prior to activation to enhance the inhibitory signal (FIG. 32E). As Siglec-9 engagement can induce apoptosis in neutrophils (von Gunten et al., Blood, 106:1423-31, 2005), PBCMC survival after Siglec-9 engagement with anti-Siglec-9 antibodies was examined. As shown in FIG. 33, there was not significant decrease in PBCMC viability at any timepoint tested in cells treated with anti-Siglec-9 antibodies or isotype control conditions.

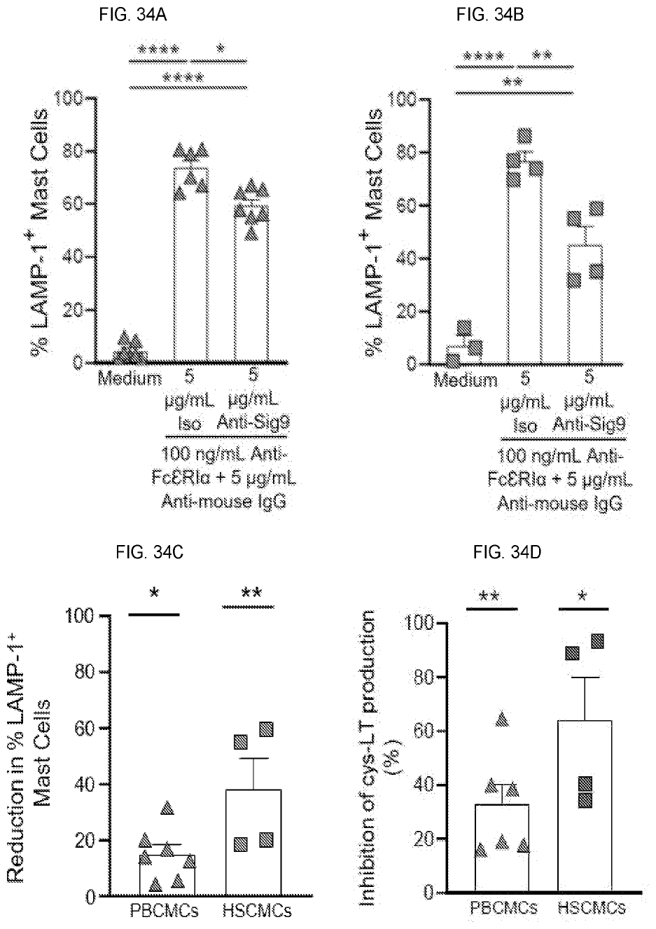

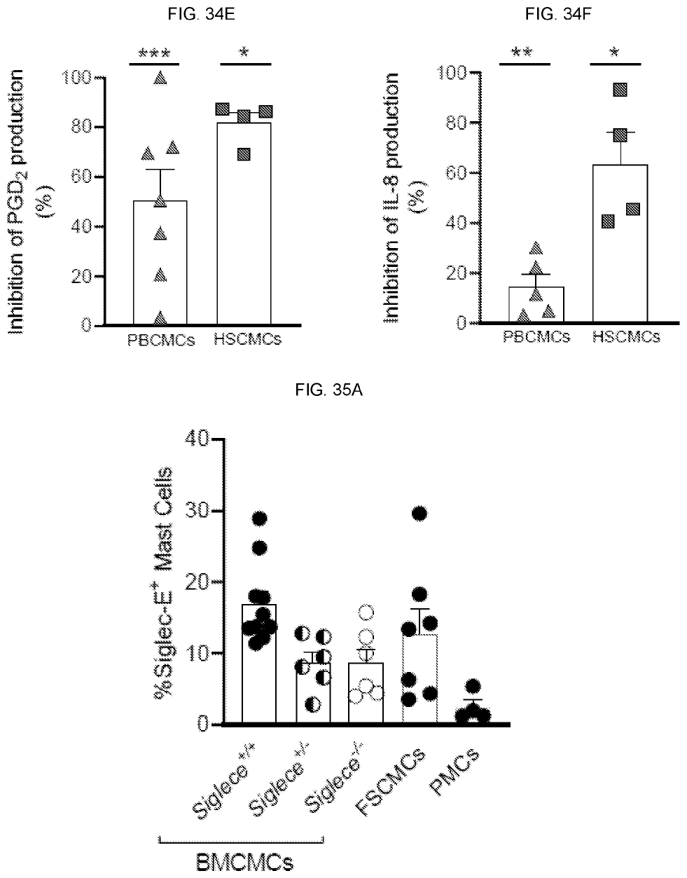

Due to the lack of anti-Siglec-9 inhibitory effect on human primary mast cell activation, whether co-engaging Siglec-9 with FcεRI could enhance Siglec-9 inhibitory function as shown for Siglec-3, -6, -7 and -8 on mast cells (Robida et al., Cells, 11, 2022; Mizrahi et al., J Allergy Clin Immunol, 134:230-3, 2014; Duan et al., J Clin Invest, 129:1387-401, 2019; Duan et al., J Immunol, 206:2290-300, 2021) was assessed. For this purpose, Siglec-9 and FcεRI on PBCMCs and HSCMC were co-aggregated using a secondary cross-linking antibody that recognizes both anti-Siglec-9 and anti-FcεRIα antibodies. A significant reduction in PBCMC (FIG. 34A) and HSCMC (FIG. 34B) activation after Siglec-9 co-engagement that was even more pronounced in HSCMCs than in PBCMCs (FIG. 34C) was observed. Cross-linking of Siglec-9 with FcεRIα also resulted in reduced production of the arachidonic acid metabolites cys-leukotrienes (cys-LT) (FIG. 34D) and prostaglandin (PG)D₂ (FIG. 34E) in both PBCMC and HSCMC. Finally, the effect of Siglec-9-FcεRI co-engagement on mast cells' ability to generate chemokines upon IgE-dependent activation was tested. As shown in FIG. 34F, both PBCMC and HSCMC released significant reduced amounts of IL-8 when Siglec-9 was cross-engaged with FcεRIα. Moreover, HSCMCs but not PBCMCs were inhibited in their ability to release MCP-1 upon Siglec-9 and FcεRI co-engagement (46.5%±12.5% inhibition, P<0.005 vs. cells treated with isotype control, n=6 experiments with cells obtained from individual donors). Overall, this data suggests that Siglec-9 proximity to FcεRI increases the effectiveness of the Siglec-9 mediated inhibitory signal.

(E) Discussion. In this Example, human mast cells expressing functional Siglec-9 is reported. Specifically, it is shown that Siglec-9 can inhibit human mast cell degranulation upon engagement with Siglec-9 ligands, GlycA and HA-HMW. Moreover, it was observed that co-engagement of Siglec-9 with FcεRI can reduce human mast cell degranulation and limit their ability to produce arachidonic acid metabolites, MCP-1, and IL-8.

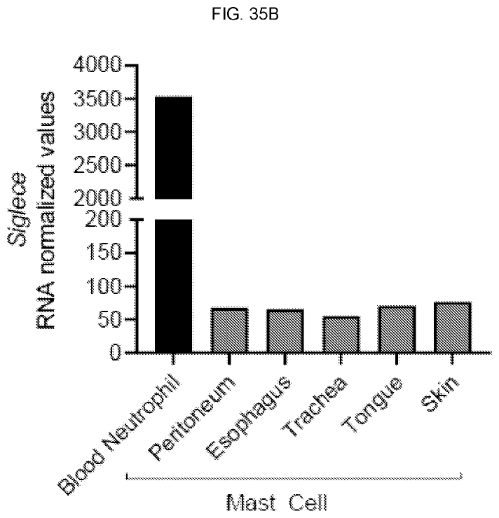

Prior studies reported that freshly isolated human skin (Duan et al., J Clin Invest, 129:1387-401, 2019) and lung (Ronnberg et al., Front Immunol, 12:804812, 2021) mast cells express surface Siglec-9. In the current study, it was confirmed that Siglec-9 is expressed in freshly isolated human lung mast cells, HSCMCs, and PBCMCs (FIG. 22A). The levels of Siglec-9 mRNA and protein expression were comparable to those found for Siglec-7 and Siglec-8 in the human mast cells tested (FIGS. 23A-23C). Previous studies showed that cord blood cultured mast cells and PBCMC do not express mRNA for Siglec-9 (Yokoi et al., Allergy, 61:769-76, 2006). The discrepancy between this data and the findings from previous studies cannot yet be fully explained. However, it was observed that the kinetics of expression of Siglec-9 parallels the expression of FcεRI in CD34⁺ cells as they differentiate into mast cells in vitro (FIG. 22C). This observation suggests that variations in the culture conditions for CD34⁺-derived cultured mast cells may affect the levels of Siglec-9 expression levels detected by different laboratories. The correlation between Siglec-9 expression and cell maturation is not unique to mast cells as it has been noted previously in other immune cells like NK cells (Jandus et al., J Clin Invest, 124:1810-20, 2014). Importantly, Siglec-6 and Siglec-8 expression also matches an increase in CD51 and FcεRIα expression and histamine content in PBCMCs (Yokoi et al., Allergy, 61:769-76, 2006) suggesting that mast cells upregulate the expression of a set of inhibitory receptors capable to engage an array of potential Siglec ligands during maturation to maintain mast cell quiescence. This hypothesis was addressed herein in part by showing that Siglec-9 deletion by a CRISPR-editing approach led to an increase in LAMP-1 expression in LAD2 cells at steady state (FIG. 27C). Moreover, Siglec-9-deleted LAD2 cells were more susceptible to IgE-dependent and IgE-independent stimuli (FIGS. 27D-27F). Currently, there are no reports on how Siglec-9 deficiency can alter the function of human immune cells. However, studies using mice deficient in Siglec-E, the murine homologue of Siglec-9, suggest that Siglec-E-deficiency can impact the activation status of immune cells as observed for Siglec-9-deficient mast cells. For example, it has been shown that microglia from Siglec-E-deficient mice displays aggravated pro-inflammatory characteristics when exposed to neural debris damage-associated molecular patterns (DAMPs) (Claude et al., J Neurosci, 33:18270-6, 2013) or lipopolysaccharide (LPS) (Li et al., J Neuroinflammation, 19:191, 2022). Similarly, Siglec-E-deficient neutrophils exhibited increased ability to migrate to the lung in an acute lung airway inflammation model induced by aerosolized LPS (McMillan et al., Blood, 121:2084-94, 2013). Herein, whether Siglec-E-deficiency can impact mast cell function could not be examined as it was found that bone marrow-derived cultured mast cells (BMCMCs), peritoneal mast cells, and fetal skin-derived cultured mast cells showed very low surface expression of Siglec-E (FIG. 35A). These observations are in agreement with the RNAseq database The from Immunological Genome Project (ImmGen, http://rstats.immgen.org/Skyline/skyline.html) showing that mast cells from the peritoneum, esophagus, trachea, tongue, or skin express very low gene expression levels of Siglec-E (FIG. 35B) when compared with blood neutrophils.

Siglec ligands can act in trans, on adjacent cells, and in cis, where they cluster Siglecs on the same cell's membrane and maintain a basal level of inhibitory signaling that increases the threshold for immune cell activation. Depletion of Siglec ligands in cis by sialidases or oxidative cleavage has been linked to increased activity of B cells (Courtney et al., Proc Natl Acad Sci USA, 106:2500-5, 2009), macrophages (Haney et al., Nat Genet, 50:1716-27, 2018), microglia (Pluvinage et al., Nature, 568:187-92, 2019), and monocyte-derived dendritic cells (Silva et al., Oncotarget, 7:41053-66, 2016), and prolonged inhibition of sialic acid biosynthesis renders phagocytes more prone to activation (Bull et al., Immunol Cell Biol, 95:408-15, 2017). The increased expression of activation markers in Siglec-9-deleted LAD2 cells points to a possible role for Siglec-9 interactions with sialic acid in cis in mast cell homeostasis. Previously, the ligands for Siglec-9 had only been found on endothelial cells (Aalto et al., Blood, 118:3725-33, 2011), red blood cells (Lizcano et al., Blood, 129:3100-10, 2017), the upper airway (Jia et al., J Allergy Clin Immunol, 135:799-810 e7, 2015), and the human aorta (Zhang et al., Life Sci, 216:189-99, 2019). In this Example, it is demonstrated that human mast cells express ligands for Siglec-9 (FIGS. 27G & 29A). Also reported herein is that human mast cells highly express mRNA for enzymes involved in sialic acid biosynthesis and sialylation (FIGS. 29A-29F). Importantly, sialic acid removal in LAD2 cells, and to a lesser degree in HSCMCs, led to an increase in the expression of mast cell activation markers as it was observed in Siglec-9-deleted mast cells (FIGS. 27J-27K) supporting the hypothesis that Siglec-9 interactions with sialic acids in cis contribute to mast cell quiescence.

Despite the progress made to identify Siglec ligands, their carrier proteins and their tissue expression patterns, the identities and physiological roles of endogenous Siglec ligands relevant to mast cell biology are unknown. Using synthetic glycan arrays, it has been shown that Siglec-9 binds preferentially to the sulfated sialylated trisaccharide structure (NeuAc α2,3 Gal β1,4 6-SO₃-GlcNAc). The search for endogenous ligands with the sulfated sialylated trisaccharide structure required for binding to Siglec-9 has been most successful for this receptor.

In human upper airway tissues, Siglec-9 can bind to the glycans of the mucin MUC5B (Jia et al., J Allergy Clin Immunol, 135:799-810 e7, 2015).

Siglec-9 can also bind the erythrocyte sialoglycoprotein, GlycA. Importantly, Siglec-9-GlycA interactions have been shown to have immunosuppressive effects on neutrophils including decreased degranulation, reactive oxygen species (ROS) and neutrophil extracellular trap (NET) production, chemotaxis, and bacterial killing (Lizcano et al., Blood, 129:3100-10, 2017). Siglec-9 mediated inhibition of neutrophils was also observed after treatment with the glycosaminoglycan, HMW-HA (Secundino et al., J Mol Med (Berl), 94:219-33, 2016). Based on this evidence, GlycA and HMW-HA effects on mast cell function were investigated. It was observed that mast cell treatment with either GlycA or HMW-HA, was able to decrease mast cell activation by IgE-dependent or IgE-independent stimuli (FIG. 24A). Although mast cells express CD44, the primary receptor of HMW-HA (Fukui et al., Clin Immunol, 94:173-8, 2000), the inhibitory effect of HMW-HA was specific to Siglec-9 in this system since LAD2 cells deficient in Siglec-9 showed no decrease in their ability to degranulate when activated in the presence of HMW-HA (FIG. 31). Overall, this data demonstrates that Siglec-9 and its ligands, whether acting in trans or cis, play an important role in maintaining mast cell quiescence and limiting their activation.

Siglecs are endocytic receptors that either constitutively cycle between the cell surface and intracellular endosomes, or can undergo endocytosis upon ligation by antibody or multivalent ligands (Macauley et al., Nat Rev Immunol, 14:653-66, 2014). Compared to the endocytic capacity of Siglec-6 and Siglec-8 on human skin mast cells (Robida et al., Cells, 11, 2022), the internalization kinetics of Siglec-9 closely resembled Siglec-6, with most of the receptor remaining on the cell surface at 24 h after Siglec-9 ligation. Contrastingly, 50% of Siglec-8 is endocytosed at 2 h post engagement and almost none of the receptor could be detected on mast cell surface at 24 h post engagement (Robida et al., Cells, 11, 2022).

The use of antibodies against Siglecs (Landolina et al., Pharmacol Res, 158:104682, 2020; Schanin et al., Mucosal Immunol, 14:366-76, 2021; Youngblood et al., JCI Insight, 4, 2019; Kerr et al., Clin Exp Allergy, 50:904-14, 2020; Gebremeskel et al., Front Immunol, 12:650331, 2021) and glycomimetic Siglec ligands on multivalent scaffolds, like nanoparticle, liposomes, or polymer, that aggregate Siglec receptors (Robida et al., Cells, 11, 2022; Delaveris et al., ACS Cent Sci, 7:650-7, 2021; Chen et al., PLOS One, 7: e39039, 2012) has demonstrated that Siglec engagement can be leveraged to modulate immune and inflammatory responses. Here, it is reported that Siglec-9 co-engagement with FcεRI can reduce human mast cell degranulation, arachidonic acid production, and chemokine release (FIGS. 34A-34F). Similarly, several studies have demonstrated that clustering of CD33 (Duan et al., J Clin Invest, 129:1387-401, 2019), Siglec-6 (Robida et al., Cells, 11, 2022), Siglec-7 (Mizrahi et al., J Allergy Clin Immunol, 134:230-3, 2014), or Siglec-8 (Yokoi et al., J Allergy Clin Immunol, 121:499-505 e1, 2008) with FcεRI is important for optimal inhibition of mast cells responses in vitro. Prior studies have shown that several CD33-related Siglecs, namely CD33, Siglec-6, Siglec-7, and Siglec-8, can prevent the release of mast cell mediators, mast cell-dependent anaphylaxis or inflammation in mouse models of disease (Bochner et al., Mol Aspects Med, 101104, 2002).

The addition of Siglec-9 to the repertoire of inhibitory receptors that can modulate mast cell function is significant for two reasons. First, mast cells may exhibit differential responsiveness to Siglec modulation depending on the tissue where they reside or the inflammatory microenvironment that may change over time. Accordingly, a broad repertoire of functional Siglecs may be needed for mast cell targeting in different conditions. The rationale for this is that inflammatory environmental changes are known to affect the expression of Siglecs and Siglec ligands. For example, it has been shown that patients with COPD (Zeng et al., Sci Rep, 7:10116, 2017), chronic rhinosinusitis (Jia et al., J Allergy Clin Immunol, 135:799-810 e7, 2015), and rheumatoid arthritis (Wang et al., Scand J Immunol, 85:433-40, 2017) exhibited an up-regulation in the expression of Siglec-9 and Siglec-9 ligands. In relation to mast cells, this Example shows that Siglec-9 inhibitory effects were more pronounced in HSCMC than in PBCMCs which may correlate to the expression levels of Siglec-9 and its ligands in these cells (FIG. 345A-34F). Second, certain Siglecs may play an important role in cell homeostasis that may preclude the use of a Siglec targeted therapy in certain conditions. For example, Siglec-6 is specifically expressed on mast cells when compared with other immune cells (Robida et al., Cells, 11, 2022), but it is also expressed on trophoblast cells of the placenta (Brinkman-Van der Linden et al., Glycobiology, 17:922-31, 2007). Importantly, placental Siglec-6 expression correlates with preterm preeclampsia (Rumer et al., Reprod Sci, 20:646-53, 2013) suggesting that Siglec-6 may contribute or represent a response to preeclampsia pathogenesis.

In summary, this study provides evidence that human mast cells express Siglec-9 with inhibitory capabilities that impact mast cell function in normal conditions and disease.

(VI) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds to” or “specifically binds” refers to an association of a binding domain to its cognate binding molecule with an affinity or Ka (i.e., an equilibrium association constant of a particular binding interaction with units of 1/M) equal to or greater than 105 M-1, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 107 M-1, at least 108 M-1, at least 109 M-1, at least 1010 M-1, at least 1011 M-1, at least 1012 M-1, or at least 1013 M-1. In particular embodiments, “low affinity” binding domains refer to those binding domains with a Ka of up to 107 M-1, up to 106 M-1, up to 105 M-1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of reducing mast cell activation comprising administering an antibody or binding fragment thereof that binds sialic acid-binding immunoglobulin-like lectin-9 (Siglec-9) on the mast cell, thereby reducing mast cell activation, wherein the antibody or binding fragment thereof comprises clone 191240, clone K8, or KALLI.

2. A method of reducing mast cell activation comprising administering a sialic acid-binding immunoglobulin-like lectin-9 (Siglec-9) ligand that binds Siglec-9 on the mast cell, thereby reducing mast cell activation.

3. The method of claim 1, wherein the Siglec-9 ligand comprises an antibody or binding fragment thereof, a sialoglycoprotein, a glycosaminoglycan, a sialyl oligosaccharide, or a mucin.

4. The method of claim 3, wherein the antibody or binding fragment thereof comprises a binding domain of clone 191240, clone K8, or KALLI.

5. The method of claim 3, wherein the antibody or binding fragment thereof is humanized.

6. The method of claim 3, wherein the sialoglycoprotein comprises glycophorin A.

7. The method of claim 3, wherein the glycosaminoglycan comprises hyaluronic acid (HA).

8. The method of claim 7, wherein the HA is a high molecular weight hyaluronic acid (HMW) HA.

9. The method of claim 3, wherein the glycosaminoglycan comprises heparin sulfate, dermatan sulfate, or keratan sulfate.

10. The method of claim 3, wherein the sialyl oligosaccharide comprises triaose, tetraose, pentose, or hexose.

11. The method of claim 3, wherein the sialyl oligosaccharide is singly or di-sialylated.

12. The method of claim 3, wherein the mucin comprises MUC5B, MUC1, or MUC16.

13. The method of claim 2, wherein the Siglec-9 ligand comprises pS9L.

14. The method of claim 2, wherein the Siglec-9 ligand is part of a multi-domain binding molecule.

15. The method of claim 14, wherein the multi-domain binding molecule comprises an FCεR binding domain.

16. The method of claim 15, wherein the FCεR binding domain comprises a binding domain of AER-37 (CRA-1) or 15.1.

17. The method of claim 16, wherein the binding domain of AER-37 (CRA-1) or 15.1 is humanized.

18. The method of claim 2, further comprising administering an antibody or binding fragment thereof that binds FCεRI.

19. The method of claim 18, wherein the an antibody or binding fragment thereof that binds FCεRI includes the binding domain of of AER-37 (CRA-1) or 15.1.

20. The method of claim 19, wherein the binding domain of AER-37 (CRA-1) or 15.1 is humanized.

21. The method of claim 2, wherein the Siglec-9 ligand is attached to a polymer, dendrimer, nanoparticle, or liposome.

22. The method of claim 2, wherein the mast cell is a human mast cell.

23. The method of claim 2, wherein the reducing mast cell activation reduces mast cell degranulation, arachidonic acid production, or chemokine release.

24. The method of claim 2, wherein the mast cell is within a subject.

25. The method of claim 24, wherein the subject is a human subject.

26. The method of claim 23, wherein the reducing ameliorates a symptom of a mast-cell associated inflammatory disorder.

27. The method of claim 26, wherein the mast-cell associated inflammatory disorder is an allergic disease, arthritis, or mastocytosis.

28. The method of claim 23, wherein the reducing treats an IgE-mediated disorder.

29. The method of claim 28, wherein the IgE-mediated disorder comprises allergic rhinitis, allergic asthma, non-allergic asthma, atopic dermatitis, allergic gastroenteropathy, anaphylaxis, urticaria, food allergy, allergic bronchopulmonary aspergillosis, parasitic disease, interstitial cystitis, hyper-IgE syndrome, ataxia-telangiectasia, Wiskott-Aldrich syndrome, athymic lymphoplasia, IgE myeloma, graft-versus-host reaction, or allergic purpura.

30. The method of claim 23, wherein the reducing decreases antigen-specific mast cell degranulation as compared to the amount of antigen-specific mast cell degranulation under comparable conditions absent the administering.

31. A composition comprising a pharmaceutically acceptable carrier and a therapeutically effective amount of a Siglec-9 ligand.

32. The composition of claim 31, wherein the Siglec-9 ligand comprises an antibody or binding fragment thereof, a sialoglycoprotein, a glycosaminoglycan, a sialyl oligosaccharide, or a mucin.

33. The composition of claim 32, wherein the antibody or binding fragment thereof comprises a binding domain of clone 191240, clone K8, or KALLI.

34. The composition of claim 32, wherein the antibody or binding fragment thereof is humanized.

35. The composition of claim 32, wherein the sialoglycoprotein comprises glycophorin A.

36. The composition of claim 32, wherein the glycosaminoglycan comprises hyaluronic acid (HA).

37. The composition of claim 36, wherein the HA is a high molecular weight hyaluronic acid (HMW) HA.

38. The composition of claim 32, wherein the glycosaminoglycan comprises heparin sulfate, dermatan sulfate, or keratan sulfate.

39. The composition of claim 32, wherein the sialyl oligosaccharide comprises triaose, tetraose, pentose, or hexose.

40. The composition of claim 32, wherein the sialyl oligosaccharide is singly or di-sialylated.

41. The composition of claim 32, wherein the mucin comprises MUC5B, MUC1, or MUC16.

42. The composition of claim 31, wherein the Siglec-9 ligand comprises pS9L.

43. The composition of claim 31, wherein the Siglec-9 ligand is part of a multi-domain binding molecule.

44. The composition of claim 43, wherein the multi-domain binding molecule comprises an FCεR binding domain.

45. The composition of claim 44, wherein the FCεR binding domain comprises a binding domain of AER-37 (CRA-1) or 15.1.

46. The composition of claim 45, wherein the binding domain of AER-37 (CRA-1) or 15.1 is humanized.

47. The composition of claim 31, wherein the Siglec-9 ligand is attached to a polymer, dendrimer, nanoparticle, or liposome.


SEQUENCE LISTING (23 sequences)

SEQ ID NO: 1 - protein, 465 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQKTSSSSSSAFEQLFPHPSIPFRAEASKYYLSGTDRNYFRLHGPHSLIAAPQICCCSKKEAIYPV

SEQ ID NO: 2 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 3 - DNA, 21 bp, synthetic construct
  source 1..21: mol_type = other DNA
gccataagtttgcagcatctc

SEQ ID NO: 4 - DNA, 21 bp, synthetic construct
  source 1..21: mol_type = other DNA
gccattggaagctctatctgc

SEQ ID NO: 5 - DNA, 22 bp, synthetic construct
  source 1..22: mol_type = other DNA
gttgggggtgaagtcagaaaag

SEQ ID NO: 6 - DNA, 20 bp, synthetic construct
  source 1..20: mol_type = other DNA
ggtgggaatctggatgagtt

SEQ ID NO: 7 - DNA, 20 bp, synthetic construct
  source 1..20: mol_type = other DNA
aatctgacctgctctgtgcc

SEQ ID NO: 8 - DNA, 20 bp, synthetic construct
  source 1..20: mol_type = other DNA
aagttctgaggcgggtagga

SEQ ID NO: 9 - DNA, 20 bp, synthetic construct
  source 1..20: mol_type = other DNA
ccgctgtggtcatttaggaa

SEQ ID NO: 10 - DNA, 20 bp, synthetic construct
  source 1..20: mol_type = other DNA
tccatctctggtccccaaat

SEQ ID NO: 11 - DNA, 20 bp, synthetic construct
  source 1..20: mol_type = other DNA
ccgctgtggtcatttaggaa

SEQ ID NO: 12 - DNA, 20 bp, synthetic construct
  source 1..20: mol_type = other DNA
ggggtgagctagagtgacta

SEQ ID NO: 13 - DNA, 20 bp, synthetic construct
  source 1..20: mol_type = other DNA
ctcccgggaagacagttttt

SEQ ID NO: 14 - DNA, 20 bp, synthetic construct
  source 1..20: mol_type = other DNA
gtaagcagatggcgtcttga

SEQ ID NO: 15 - DNA, 20 bp, synthetic construct
  source 1..20: mol_type = other DNA
ctctgtggctgctcttgtca

SEQ ID NO: 16 - DNA, 20 bp, synthetic construct
  source 1..20: mol_type = other DNA
tggtgaggaggagggagatg

SEQ ID NO: 17 - DNA, 18 bp, synthetic construct
  source 1..18: mol_type = other DNA
ctgctccctcctctgctc

SEQ ID NO: 18 - DNA, 19 bp, synthetic construct
  source 1..19: mol_type = other DNA
ccggcagaaaccaaaggaa

SEQ ID NO: 19 - DNA, 20 bp, synthetic construct
  source 1..20: mol_type = other DNA
tgacggcgtctggaactcta

SEQ ID NO: 20 - DNA, 20 bp, synthetic construct
  source 1..20: mol_type = other DNA
gtagcaacacaaacccgcag

SEQ ID NO: 21 - DNA, 20 bp, synthetic construct
  source 1..20: mol_type = other DNA
gagtcaacggatttggtcgt

SEQ ID NO: 22 - DNA, 20 bp, synthetic construct
  source 1..20: mol_type = other DNA
ttgattttggagggatctcg

SEQ ID NO: 23 - RNA, 20 nt, synthetic construct
  source 1..20: mol_type = other RNA
gacgatgcagagttccgtga